SEQUENCE LISTING (36 sequences)

SEQ ID NO: 1 - protein, 10 aa, synthetic construct
GRPFSNYGMG

SEQ ID NO: 2 - protein, 10 aa, synthetic construct
GISWSGGSTD

SEQ ID NO: 3 - protein, 17 aa, synthetic construct
VFSGAETAYYPSDDYDY

SEQ ID NO: 4 - protein, 5 aa, synthetic construct
NYGMG

SEQ ID NO: 5 - protein, 17 aa, synthetic construct
GISWSGGSTDYADSVKG

SEQ ID NO: 6 - protein, 17 aa, synthetic construct
VFSGAETAYYPSDDYDY

SEQ ID NO: 7 - protein, 7 aa, synthetic construct
GRPFSNY

SEQ ID NO: 8 - protein, 6 aa, synthetic construct
SWSGGS

SEQ ID NO: 9 - protein, 17 aa, synthetic construct
VFSGAETAYYPSDDYDY

SEQ ID NO: 10 - protein, 8 aa, synthetic construct
GRPFSNYG

SEQ ID NO: 11 - protein, 8 aa, synthetic construct
ISWSGGST

SEQ ID NO: 12 - protein, 19 aa, synthetic construct
AAVFSGAETAYYPSDDYDY

SEQ ID NO: 13 - protein, 6 aa, synthetic construct
SNYGMG

SEQ ID NO: 14 - protein, 13 aa, synthetic construct
FVAGISWSGGSTD

SEQ ID NO: 15 - protein, 18 aa, synthetic construct
AAVFSGAETAYYPSDDYD

SEQ ID NO: 16 - protein, 10 aa, synthetic construct
GSIFSIFDMG

SEQ ID NO: 17 - protein, 9 aa, synthetic construct
RITNGGITN

SEQ ID NO: 18 - protein, 15 aa, synthetic construct
DIAANIGSNILYDDY

SEQ ID NO: 19 - protein, 5 aa, synthetic construct
IFDMG

SEQ ID NO: 20 - protein, 16 aa, synthetic construct
RITNGGITNYLDSVKG

SEQ ID NO: 21 - protein, 15 aa, synthetic construct
DIAANIGSNILYDDY

SEQ ID NO: 22 - protein, 7 aa, synthetic construct
GSIFSIF

SEQ ID NO: 23 - protein, 5 aa, synthetic construct
TNGGI

SEQ ID NO: 24 - protein, 15 aa, synthetic construct
DIAANIGSNILYDDY

SEQ ID NO: 25 - protein, 8 aa, synthetic construct
GSIFSIFD

SEQ ID NO: 26 - protein, 7 aa, synthetic construct
ITNGGIT

SEQ ID NO: 27 - protein, 17 aa, synthetic construct
YADIAANIGSNILYDDY

SEQ ID NO: 28 - protein, 6 aa, synthetic construct
SIFDMG

SEQ ID NO: 29 - protein, 12 aa, synthetic construct
LVARITNGGITN

SEQ ID NO: 30 - protein, 16 aa, synthetic construct
YADIAANIGSNILYDD

SEQ ID NO: 31 - protein, 126 aa, synthetic construct
EVQLVESGGGLVQAGGSLRLSCAASGRPFSNYGMGWFRQAPGKKREFVAGISWSGGSTDYADSVKGRFTISRDNAKNTVYLQMNSLKPEDTAVYYCAAVFSGAETAYYPSDDYDYWGQGTQVTVSS

SEQ ID NO: 32 - protein, 123 aa, synthetic construct
QVQLVESGGGLVQAGGSLRLSCEASGSIFSIFDMGWYRRPPGAQRELVARITNGGITNYLDSVKGRFSISRDNAKNTVYLQMNSLNAEDTAVYYCYADIAANIGSNILYDDYWGQGTQVTVSS

SEQ ID NO: 33 - protein, 358 aa, synthetic construct
EVQLVESGGGLVQAGGSLRLSCAASGRPFSNYGMGWFRQAPGKKREFVAGISWSGGSTDYADSVKGRFTISRDNAKNTVYLQMNSLKPEDTAVYYCAAVFSGAETAYYPSDDYDYWGQGTQVTVSSEPKSSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 355 aa, synthetic construct
QVQLVESGGGLVQAGGSLRLSCEASGSIFSIFDMGWYRRPPGAQRELVARITNGGITNYLDSVKGRFSISRDNAKNTVYLQMNSLNAEDTAVYYCYADIAANIGSNILYDDYWGQGTQVTVSSEPKSSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 35 - protein, 358 aa, synthetic construct
EVQLVESGGGLVQAGGSLRLSCAASGRPFSNYGMGWFRQAPGKKREFVAGISWSGGSTDYADSVKGRFTISRDNAKNTVYLQMNSLKPEDTAVYYCAAVFSGAETAYYPSDDYDYWGQGTQVTVSSEPKSSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 36 - protein, 355 aa, synthetic construct
QVQLVESGGGLVQAGGSLRLSCEASGSIFSIFDMGWYRRPPGAQRELVARITNGGITNYLDSVKGRFSISRDNAKNTVYLQMNSLNAEDTAVYYCYADIAANIGSNILYDDYWGQGTQVTVSSEPKSSDKTHTCPPCPAPEAAGGPSVFLFPPKPKDTLYITREPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2026/0103515 A1
(43) Pub. Date: Apr. 16, 2026

(54) DOSES AND PHARMACEUTICAL COMPOSITIONS OF A CD33 x Vd2 BISPECIFIC ANTIBODY FOR THE TREATMENT OF CANCER

(71) Applicant: Janssen Biotech, Inc., Horsham, PA (US)

(72) Inventors: Ranjeet Prasad DASH, Raritan, NJ (US); Brian Richard McNaughton, Raritan, NJ (US); Kavita Raman, Raritan, NJ (US); Nirav Raju Shah, Raritan, NJ (US); Christina Diane Guttke, Raritan, NJ (US); Karim SAFER, Raritan, NJ (US); Lorena A. Kallal, Raritan, NJ (US); Sara Mohamed A. Elashkar, Beerse (BE); Kathryn Lee Bradford, Raritan, NJ (US)

(21) Appl. No.: 19/293,723

(22) Filed: Aug. 7, 2025

Related U.S. Application Data
(60) Provisional application No. 63/680,866, filed on Aug. 8, 2024.

(51) Int. Cl. C07K 16/28; A61K 39/00; A61K 47/18; A61K 47/26

(52) U.S. Cl. CPC C07K 16/2803; A61K 47/183; A61K 47/26; C07K 16/2809; A61K 2039/54; A61K 2039/545; C07K 2317/31

(57) ABSTRACT

The present disclosure relates to doses and dosing regimens of a bispecific antibody that binds myeloid cell surface antigen CD33, and the Vδ2 chain of the human Vγ9Vδ2 T cell receptor; to pharmaceutical compositions comprising said antibodies, as well as methods of administration of the referred doses, and to the use of said antibodies in the treatment of Acute Myeloid Leukemia (AML) or Myelodysplastic Neoplasms (MDS), in particular Relapsed or Refractory (R/R) AML or MDS.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### CROSS REFERENCE TO RELATED APPLICATIONS

This application claims priority to and the benefit of U.S. Provisional Application No. 63/680,866, filed Aug. 8, 2024, the disclosure of which is incorporated herein by reference in its entirety.

### SEQUENCE LISTING

The instant application contains a Sequence Listing XML which is being submitted herewith electronically in XML format and is hereby incorporated by reference in its entirety. Said XML copy, created on Dec. 8, 2025, is named 103693004382_SL_2.xml and is 33,990 bytes in size.

### FIELD OF THE INVENTION

The present disclosure relates to doses and dosing regimens of antibodies; pharmaceutical compositions comprising said antibodies, methods of administration of the referred doses, and the use of said antibodies in cancer therapy.

### BACKGROUND OF THE INVENTION

The CD33×Vδ2 bispecific antibody disclosed herein is a gamma-delta (γδ) T-cell engager that recognizes the Vδ2 portion of the Vγ9Vδ2 (Vgamma) Vdelta2) T-cell receptor on a subset of T-lymphocytes and the CD33 antigen on malignant myeloid blasts and mature healthy myeloid cells. This design is intended to enhance natural tumor recognition, engage Vγ9Vδ2 T-cells at the tumor, and selectively kill cancer cells, while avoiding the clinical risks due to widespread T-cell activation and cytotoxicity of healthy cells. This CD33×Vδ2 bispecific antibody is being developed for the treatment of AML or higher-risk types of MDS, which no longer responds to standard therapies.

AML is an aggressive, heterogeneous clonal disease of the blood and BM. Disease remission can be achieved with standard induction chemotherapy but relapsed or refractory (R/R) disease remains a challenge due to persistence of leukemic stem cells and represents an unmet medical need. To date, immunotherapy in AML has not been successful and has shown limited clinical activity with substantial toxicities owing to the lack of suitable target antigens.

MDS represents a heterogeneous group of malignant hematopoietic stem cell disorders that are characterized by cytopenias, myeloid dysplasia, and a risk of transformation to AML. As with AML, hematopoietic stem cell transplant (HSCT) represents the only potentially curative option with limited availability. Currently there are no approved treatment options for patients with relapsed or refractory disease. As in AML, immunotherapy in higher-risk types of MDS has not been successful and has been associated with substantial toxicity.

An objective of the present invention is to determine the safety and tolerability of the CD33×Vδ2 bispecific antibody disclosed herein.

An objective of the present invention is the identification of the recommended Phase 2 dose(s) (RP2D [s]) of the CD33×Vδ2 bispecific antibody disclosed herein, in participants with relapsed or refractory (R/R) acute myeloid leukemia (AML) or R/R higher-risk types of myelodysplastic neoplasms (MDS).

An objective of the present invention is the determination of the safety, pharmacokinetic (PK), pharmacodynamics, immunogenicity, and the preliminary clinical activity of the CD33×Vδ2 bispecific antibody monotherapy at the RP2D(s) administered in participants with R/R AML or R/R higher-risk types of MDS.

An objective of the present invention is the provision of a safe and effective amount of the CD33×Vδ2 bispecific antibody for the treatment of AML or MDS, in particular R/R AML or R/R MDS.

An objective of the present invention is the provision of a therapeutically effective amount of the CD33×Vδ2 bispecific antibody for the treatment of AML or MDS, in particular R/R AML or R/R MDS.

An objective of the present invention is the provision of a pharmaceutically acceptable amount of the CD33×Vδ2 bispecific antibody for the treatment of AML or MDS, in particular R/R AML or R/R MDS.

Another objective of the present invention is the determination of the response of malignancies with specific mutational/immunologic signatures to the treatment with the CD33×Vδ2 bispecific antibody disclosed herein.

### SUMMARY OF THE INVENTION

The disclosure relates to a unit dose of a CD33×Vδ2 bispecific antibody, wherein said unit dose is selected from the group consisting of: from 7 milligrams (mg) to 70 mg. 70 mg. 69 mg, 68 mg, 67 mg, 66 mg, 65 mg, 64 mg, 63 mg, 62 mg, 61 mg, 60 mg, 59 mg, 58 mg, 57 mg, 56 mg, 55 mg, 54 mg, 53 mg, 52 mg, 51 mg, 50 mg, 49 mg, 48 mg, 47 mg, 46 mg, 45 mg, 44 mg, 43 mg, 42 mg. 41 mg, 40 mg, 39 mg, 38 mg, 37 mg, 36 mg, 35 mg, 34 mg, 33 mg, 32 mg, 31 mg, 30 mg, 29 mg, 28 mg, 27 mg, 26 mg, 25 mg, 24 mg, 23 mg, 22 mg, 21 mg, 20 mg, 19 mg, 18 mg, 17 mg, and 16 mg, 15 mg, 14 mg, 13 mg, 12 mg, 11 mg, 10 mg, 9 mg, 8 mg, 7 mg; and wherein the CD33×Vδ2 bispecific antibody comprises the polypeptides set forth in SEQ ID NOs: 33 and 34.

The disclosure relates to a unit dose of a CD33×Vδ2 bispecific antibody, wherein said unit dose is selected from the group consisting of: from 385 milligrams (mg) to 0.017 mg, 385 mg, 200 mg, 110 mg, 105 mg, 100 mg, 90 mg, 80 mg, 70 mg, 60 mg, 59 mg, 58 mg, 57 mg, 56 mg, 55 mg, 54 mg, 53 mg, 52 mg, 51 mg, 50 mg, 49 mg, 48 mg, 47 mg, 46 mg, 45 mg, 44 mg, 43 mg, 42 mg, 41 mg, 40 mg, 39 mg, 38 mg, 37 mg, 36 mg, 35 mg, 34 mg, 33 mg, 32 mg, 31 mg, 30 mg, 29 mg, 28 mg, 27 mg, 26 mg, 25 mg, 24 mg, 23 mg, 22 mg, 21 mg, 20 mg, 19 mg, 18 mg, 17 mg, 16 mg, 15 mg, 14 mg, 13 mg, 12 mg, 11 mg, 10 mg, 9 mg, 8 mg, 7 mg, 6 mg, 5 mg, 4 mg, 3 mg, 2.5 mg, 2 mg, 1.7 mg, 1 mg, 0.800 mg, 0.730 mg, 0.600 mg, 0.540 mg, 0.210 mg, 0.200 mg, 0.170 mg, 0.060 mg, 0.054 mg, 0.020 mg, and 0.017 mg; and
wherein a first antigen binding region of the CD33×Vδ2 bispecific antibody, binds the Vδ2 chain of the human Vγ9Vδ2 T cell receptor, and comprises the HCDR1, the HCDR2, and the HCDR3, of:
   a. SEQ ID NOs: 1, 2, and 3 (according to AbM), respectively;
   b. SEQ ID NOs: 4, 5, and 6 (according to Kabat), respectively;
   c. SEQ ID NOs: 7, 8, and 9 (according to Chothia), respectively;
   d. SEQ ID NOs: 10, 11, and 12 (according to IMGT), respectively; or
   e. SEQ ID NOs: 13, 14, and 15 (according to Contact), respectively; and
wherein a second antigen binding region of the CD33×Vδ2 bispecific antibody, binds CD33 and comprises the HCDR1, HCDR2 and HCDR3 of:
   a. SEQ ID NOs: 16, 17, and 18 (according to AbM), respectively;
   b. SEQ ID NOs: 19, 20, and 21 (according to Kabat), respectively;
   c. SEQ ID NOs: 22, 23, and 24 (according to Chothia), respectively;
   d. SEQ ID NOs: 25, 26, and 27 (according to IMGT), respectively; or
   e. SEQ ID NOs: 28, 29, and 30 (according to Contact), respectively.

The first antigen binding region comprises a sequence having at least 90%, 92%, 94%, 96%, 98%, or 100% sequence identity to the single chain moiety (VHH) of SEQ ID NO: 31; and the second antigen binding region comprises a sequence having at least 90%, 92%, 94%, 96%, 98%, or 100% sequence identity to the single chain moiety (VHH) of SEQ ID NO: 32.

The CD33×Vδ2 bispecific antibody comprises the polypeptides set forth in SEQ ID NOs: 33 and 34.

In one embodiment, the CD33×Vδ2 bispecific antibody comprises the polypeptides set forth in SEQ ID NOs: 35 and 36.

The unit dose of the CD33×Vδ2 bispecific antibody is comprised in a pharmaceutical composition at an antibody concentration of 2 mg/mL, 20 mg/mL, 30 mg/mL, or 70 mg/mL.

The unit dose of the CD33×Vδ2 bispecific antibody is adapted for delivery once every two weeks (Q2W), or once every week (Q1W).

The unit dose of the CD33×Vδ2 bispecific antibody is adapted for intravenous delivery.

The unit dose of the CD33×Vδ2 bispecific antibody is adapted for delivery by infusion during 0.5-4 hours, or about 1 hour.

The disclosure further relates to a pharmaceutical composition comprising: (i) the unit dose of the CD33×Vδ2 bispecific antibody presented herein, and (ii) a pharmaceutically acceptable carrier.

In the pharmaceutical composition, the CD33×Vδ2 bispecific antibody is present at a concentration of 2 mg/mL, 20 mg/mL, 30 mg/mL, or 70 mg/mL.

In the pharmaceutical composition, the pharmaceutically acceptable carrier comprises:
a buffer present at a concentration within the range from 10 mM to 80 mM, wherein the buffer comprises histidine, acetate, citrate, or phosphate;
a tonicifying agent present at a concentration within the range from 2 to 10% (w/v), wherein the tonicifying agent is one or more sugar and/or polyol comprising sorbitol, sucrose, trehalose, glycerol, mannitol, or dextrose;
a surfactant present at a concentration within the range from 0.04 to 0.2% (w/v), wherein the surfactant comprises polysorbate 20, polysorbate 80, or poloxamer 188;
a stabilizer present at a concentration within the range from 20 to 120 mM, wherein the stabilizer is L-methionine;
wherein the formulation has a pH within the range from 5 to 8 when in aqueous form.

In the pharmaceutical composition, the pharmaceutically acceptable carrier comprises:
50 mM histidine;
7.5% (w/v) sorbitol;
0.06% (w/v) polysorbate 20;
100 mM of L-methionine;
wherein the formulation has a pH of 6 when in aqueous form.

The pharmaceutical composition is adapted for intravenous administration.

The pharmaceutical composition is in the form of a vial, syringe, syringe pump, infusion pump, or an intravenous bag.

The disclosure further relates to the unit dose of the CD33×Vδ2 bispecific antibody or the pharmaceutical composition comprising said unit dose, for use in a method of treating Acute Myeloid Leukemia (AML) or Myelodysplastic Neoplasms (MDS).

The disclosure further relates to a method of treating AML or MDS in a patient, said method comprising administering to the patient in need thereof the unit dose of the CD33×Vδ2 bispecific antibody or the pharmaceutical composition comprising said unit dose, as disclosed herein.

The AML or MDS are relapsed or refractory (R/R) AML or R/R higher-risk types of MDS.

The unit dose is administered once every two weeks (Q2W), or once every week (Q1W).

The unit dose is administered intravenously.

The unit dose is administered by infusion during 0.5-4 hours, in particular about 1 hour.

The unit dose of the CD33×Vδ2 bispecific antibody is administered until progression, relapse, refractory response, intolerable toxicity, or continuous clinical benefit.

### DESCRIPTION OF THE FIGURES

FIG. 1: Simulated mean PK profile for the CD33×Vδ2 bispecific antibody serum concentrations in humans following a single intravenous (IV) dose of 0.017 mg. Dotted line indicates projected serum antibody Cₘₐₓ following a single IV dose at 0.017 mg in humans.

FIG. 2: Schematic overview of the study. * PK/PD expansion cohort(s) may be evaluated before or after the start of Part 2. More than one RP2D could be explored in Part 2. Part 2 cohorts may be revised as supported by emerging data.

### DETAILED DESCRIPTION OF THE INVENTION

### Definitions

As used in this specification and the appended claims, the singular forms “a,” “an,” and “the” include plural referents unless the content clearly dictates otherwise. Thus, for example, reference to “a cell” includes a combination of two or more cells, and the like.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies (e.g., VHH), domain antibodies and any other modified configuration of the immunoglobulin molecule that comprises an antigen binding site of the required specificity. A heavy chain is comprised of a heavy chain variable region (VH) and a heavy chain constant region (comprised of domains CH1, hinge. CH2 and CH3). A light chain, if present, is comprised of a light chain variable region (VL) and a light chain constant region (CL). The VH and the VL regions may be further subdivided into regions of hypervariability, termed complementarity determining regions (CDR). 20) interspersed with framework regions (FR). A VH or VL is composed of three CDRs and four FR segments, arranged from amino-to-carboxy-terminus in the following order: FR1, CDR1, FR2, CDR2, FR3, CDR3 and FR4, Immunoglobulins may be assigned to five major classes. IgA, IgD, IgE, IgG and IgM, depending on the heavy chain constant domain amino acid sequence. IgA and IgG are further sub-classified as the isotypes IgAQ1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Antigen” refers to any molecule (e.g., protein, peptide, polysaccharide, glycoprotein, glycolipid, nucleic acid, portions thereof, or combinations thereof) capable of being bound by an antigen binding domain or a T-cell receptor that is capable of mediating an immune response. Exemplary immune responses include antibody production and activation of immune cells, such as T cells, B cells or NK cells. Antigens may be expressed by genes, synthetized, or purified from biological samples such as a tissue sample, a tumor sample, a cell or a fluid with other biological components, organisms, subunits of proteins/antigens, killed or inactivated whole cells or lysates.

“Antigen binding region” or “antigen binding domain” or “antigen binding site” or “antigen binding fragment” refers to a portion of the antibody that binds an antigen, in particular it refers to one or more CDR regions, more in particular 2 CDR regions, more in particular 3 CDR regions. Antigen binding regions may be synthetic, enzymatically obtainable or genetically engineered polypeptides and include portions of an immunoglobulin that bind an antigen, such as the VH, the VL, the VH and the VL, Fab, Fab′, F(ab′)₂, Fd and Fv fragments, single domain antibodies (dAb) consisting of one VH domain or one VL domain, shark variable IgNAR domains, camelized VH domains, single chain moieties or single chain antibodies such as VHH domains, minimal recognition units consisting of the amino acid residues that mimic the CDRs of an antibody, such as FR3-CDR3-FR4 portions, the HCDR1, the HCDR2 and/or the HCDR3, optionally the LCDR1, the LCDR2 and/or the LCDR3, alternative scaffolds that bind an antigen, and multispecific proteins comprising the antigen binding regions. Antigen binding domains (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding regions may also be conjugated to other antibodies, proteins, antigen binding fragments or alternative scaffolds which may be monospecific or multispecific to engineer bispecific and multispecific proteins.

“Bispecific” refers to a molecule (such as an antibody) that specifically binds two distinct antigens or two distinct epitopes within the same antigen. The bispecific molecule may have cross-reactivity to other related antigens, for example to the same antigen from other species (homologs), such as human, monkey, or ape, for example Macaca fascicularis (cynomolgus monkey, cyno) or Pan troglodytes, or may bind an epitope that is shared between two or more distinct antigens.

“Complementarity determining regions” (CDR) are antibody regions that bind an antigen. There are three CDRs in the VH (HCDR1, HCDR2, HCDR3) and three CDRs in the VL, if present. (LCDR1, LCDR2, LCDR3). CDRs may be defined using various delineations such as Kabat (Wu et al. (1970) J Exp Med 132:211-50); Kabat et al., Sequences of Proteins of Immunological Interest. 5th Ed. Public Health Service. National Institutes of Health. Bethesda. Md., 1991); Chothia (Chothia et al. (1987) J Mol Biol 196:901-17); IMGT (Lefranc et al. (2003) Dev Comp Immunol 27:55-77); AbM (Martin and Thornton J Bmol Biol 263:800-15, 1996); or Contact, which is based on an analysis of the available complex crystal structures (MacCallum, R. M., Martin. A. C. R, and Thornton. J. T. “Antibody-antigen interactions: Contact analysis and binding site topography” J. Mol. Biol. 262:732-745). The correspondence between the various delineations and variable region numbering is described (see e.g. Lefranc et al. (2003) Dev Comp Immunol 27:55-77; Honegger and Pluckthun. J Mol Biol (2001) 309:657-70; International ImMunoGeneTics (IMGT) database; Web resources, www.imgt.org). Available programs such as abYsis by UCL Business PLC may be used to delineate CDRs. The terms “CDR”, “CDR1”, “CDR2”, “CDR3”, “HCDR1”, “HCDR2”, “HCDR3”, “LCDR1”, “LCDR2” and “LCDR3” as used herein includes CDRs defined by any of the methods described supra. Kabat. Chothia. IMGT, AbM, or Contact, unless otherwise explicitly stated in the specification.

The terms “first” and “second” antigen-binding regions when used herein do not refer to their orientation/position in the antibody, i.e., they have no meaning with regard to the N- or C-terminus. The terms “first” and “second” only serve to refer correctly and consistently to the two different antigen binding regions in the claims and the description.

The term “CD33” when used herein, refers to human CD33, the sequence of which is set forth in UniProtKB-P20138.

The term “human Vδ2”, when used herein, refers to the rearranged 82 chain of the Vγ9Vδ2-T cell receptor (TCR). GenBank: CAA51166.1, gives an example of a 82 sequence. TRDV2. T cell receptor delta variable 2, represents the variable region (UniProtKB-A0JD36 (A0JD36_HUMAN) gives an example of a TRDV2 sequence). “binding the Vò2 chain of a Vγ9Vδ2-TCR” means that the antibody can bind the 82 chain as a separate molecule and/or as part of a Vγ9Vδ2-TCR (T cell Receptor). However, the antibody will not bind to the γ9 chain as a separate molecule.

The term “human Vγ9”, when used herein, refers to the rearranged γ9 chain of the Vγ9Vδ2-T cell receptor (TCR). GenBank: NG_001336.2 gives an example of a γ9 sequence. TRGV9. T cell receptor gamma variable 9 represents the variable region (UniProtKB-Q99603_HUMAN gives an example of a TRGV9 sequence).

“VHH”, “single chain antibody”, “single domain antibody”, “single chain moiety”, “dAb”, or “dAb fragment” refers to an antibody fragment composed of a VH domain (Ward et al. Nature 341:544 546 (1989)). The first and the second antigen binding regions disclosed herein may be each a single chain antibody or VHH. Single chain antibodies (sdAb, also called Nanobody®, or VHH) are well known to the skilled person, see e.g., Hamers-Casterman et al. (1993) Nature 363:446, Roovers et al. (2007) Curr Opin Mol Ther 9:327 and Krah et al. (2016) Immunopharmacol Immunotoxicol 38:21. Single chain antibodies comprise a single CDR1, a single CDR2, and a single CDR3, preferably a single HCDR1, a single HCDR2, and a single HCDR3. Examples of single chain antibodies are variable fragments of heavy-chain-only antibodies, antibodies that naturally do not comprise light chains, single chain antibodies derived from conventional antibodies, and engineered antibodies. Single chain antibodies may be derived from any species including mouse, human, camel, llama, shark, goat, rabbit, and cow. For example, naturally occurring VHH molecules can be derived from antibodies raised in Camelidae species, for example in camel, dromedary, llama, alpaca and guanaco. Like a whole antibody, a single chain antibody is able to bind selectively to a single specific antigen. Single chain antibodies may contain only the variable domains of an immunoglobulin chain, i.e., CDR1, CDR2 and CDR3 and framework regions.

“Host cell” refers to any cell that contains a heterologous nucleic acid. An exemplary heterologous nucleic acid is a vector (e.g., an expression vector).

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks. CDRs, or the constant regions. Typically. “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Operatively linked” and similar phrases, when used in reference to nucleic acids or amino acids, refers to the operational linkage of nucleic acid sequences or amino acid sequence, respectively, placed in functional relationships with each other. For example, an operatively linked promoter, enhancer elements, open reading frame. 5′ and 3′ UTR, and terminator sequences result in the accurate production of a nucleic acid molecule (e.g., RNA) and in some instances to the production of a polypeptide (i.e., expression of the open reading frame). Operatively linked peptide refers to a peptide in which the functional domains of the peptide are placed with appropriate distance from each other to impart the intended function of each domain.

“Expression vector” refers to a vector that can be utilized in a biological system or in a reconstituted biological system to direct the translation of a polypeptide encoded by a polynucleotide sequence present in the expression vector.

In the context of the present disclosure. “competition” or “able to compete” or “competes” refers to any detectably significant reduction in the propensity for a particular binding molecule (e.g., an antibody) to bind a particular binding partner (e.g., the target) in the presence of another molecule (e.g., a different antibody binding the same target) that binds the binding partner. Typically, competition means an at least about 25 percent reduction, such as an at least about 50 percent, e.g., an at least about 75 percent, such as an at least 90 percent reduction in binding. 20) caused by the presence of another molecule, such as an antibody, as determined by, e.g., ELISA analysis or flow cytometry using sufficient amounts of the two or more competing molecules, e.g., antibodies. Additional methods for determining binding specificity by competitive inhibition may be found in for instance Harlow et al., Antibodies: A Laboratory Manual. Cold Spring Harbor Laboratory Press. Cold Spring Harbor. N.Y., 1988). Colligan et al., eds., Current Protocols in Immunology. Greene Publishing Assoc, and Wiley InterScience N. Y., (1992, 1993), and Muller. Meth. Enzymol. 92, 589-601 (1983)).

“Activation” or “stimulation” or “activated” or “stimulated” refers to induction of a change in the biologic state of a cell resulting in expression of activation markers, cytokine production, proliferation or mediating cytotoxicity of target cells. Cells may be activated by primary stimulatory signals. Co-stimulatory signals can amplify the magnitude of the primary signals and suppress cell death following initial stimulation resulting in a more durable activation state and thus a higher cytotoxic capacity.

The percent “Sequence identity” between two sequences is a function of the number of identical positions shared by the sequences (i.e., % identity=number of identical positions/total number of positions×100), taking into account the number of gaps, and the length of each gap, which need to be introduced for optimal alignment of the two sequences. The percent identity between two amino acid sequences may be determined using the algorithm of E. Meyers and W. Miller (Comput. Appl. Biosci. 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J. Mol. Biol. 48:444-453 (1970)) algorithm which has been incorporated into the GAP program in the GCG software package (available at www_gcg_com), using either a Blossum 62 matrix or a PAM250 matrix, and a gap weight of 16, 14, 12, 10, 8, 6, or 4 and a length weight of 1, 2, 3, 4, 5, or 6.

“Specifically binds,” “specific binding,” “specifically binding” or “binds” refer to a proteinaceous molecule binding to, or capable of binding to, an antigen or an epitope within the antigen with greater affinity than for other antigens. Typically, the proteinaceous molecule binds to the antigen or the epitope within the antigen with an equilibrium dissociation constant (K_{D}) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein).

“Pharmaceutical composition” refers to a composition that results from combining an active ingredient and a pharmaceutically acceptable carrier.

A “stable” formulation is one in which the antibody therein essentially retains its physical stability and/or chemical stability and/or biological activity during the manufacturing process and/or upon storage. Various analytical techniques for measuring protein stability are available in the art and are reviewed in Peptide and Protein Drug Delivery. 247-301. Vincent Lee Ed., Marcel Dekker. Inc., New York. N.Y., Pubs. (1991) and Jones. A. (1993) Adv. Drug Delivery Rev. 10:29-90. For example, in one embodiment, the stability of the protein is determined according to the percentage of monomer protein in the solution, with a low percentage of degraded (e.g., fragmented) and/or aggregated protein. Preferably, the formulation is stable at room temperature (about 18° C., to 30° C.) or at 40° C., for at least 1 month and/or stable at about 2-8° C., for at least 1 year or for at least 2 years. Furthermore, the formulation is preferably stable following freezing (to, e.g., 70° C.) and thawing of the formulation, in one or more freeze/thaw cycles.

An antibody “retains its physical stability” in a pharmaceutical formulation if it shows substantially no signs of, e.g., aggregation, precipitation and/or denaturation upon visual examination of color and/or clarity, or as measured by UV light scattering or by size exclusion chromatography. Aggregation is a process whereby individual molecules or complexes associate covalently or non-covalently to form aggregates. Aggregation can proceed to the extent that a visible precipitate is formed.

Stability, such as physical stability of a formulation may be assessed by methods well-known in the art, including measurement of a sample's apparent attenuation of light (absorbance, or optical density). Such a measurement of light attenuation relates to the turbidity of a formulation. The turbidity of a formulation is partially an intrinsic property of the protein dissolved in solution and is commonly determined by nephelometry and measured in Nephelometric Turbidity Units (NTU).

The degree of turbidity, e.g., as a function of the concentration of one or more of the components in the solution, e.g., protein and/or salt concentration, is also referred to as the “opalescence” or “opalescent appearance” of a formulation. The degree of turbidity can be calculated by reference to a standard curve generated using suspensions of known turbidity. Reference standards for determining the degree of turbidity for pharmaceutical compositions can be based on the European Pharmacopeia criteria (European Pharmacopoeia. Fourth Ed., Directorate for the Quality of Medicine of the Council of Europe (EDQM). Strasbourg. France). According to the European Pharmacopeia criteria, a clear solution is defined as one with a turbidity less than or equal to a reference suspension which has a turbidity of approximately 3 according to European Pharmacopeia standards. Nephelometric turbidity measurements can detect Rayleigh scatter, which typically changes linearly with concentration, in the absence of association or nonideality effects. Other methods for assessing physical stability are well-known in the art.

An antibody “retains its chemical stability” in a pharmaceutical formulation, if the chemical stability at a given time is such that the antibody is considered to still retain its biological activity as defined below. Chemical stability can be assessed by, e.g., detecting and quantifying chemically altered forms of the antibody. Chemical alteration may involve size modification (e.g, clipping) which can be evaluated using size exclusion chromatography. SDS-PAGE and/or matrix-assisted laser desorption ionization/time-of-flight mass spectrometry (MALDI/TOF MS), for example. Other types of chemical alteration include charge alteration (e.g, occurring as a result of deamidation or oxidation) which can be evaluated by ion-exchange chromatography, for example.

An antibody “retains its biological activity” in a pharmaceutical formulation if the antibody in a pharmaceutical formulation is biologically active for its intended purpose. For example, biological activity is retained if the biological activity of the antibody in the pharmaceutical formulation is within about 30%, about 20%, or about 10% (within the errors of the assay) of the biological activity exhibited at the time the pharmaceutical formulation was prepared (e.g., as determined in an antigen binding assay).

“Pharmaceutically acceptable carrier” or “excipient” refers to an ingredient in a pharmaceutical composition or formulation, other than the active ingredient, which is nontoxic to a subject. This ingredient may be added to a formulation to provide a desired consistency, e.g., altering the bulk properties, to improve stability, and/or to adjust osmolality. Exemplary pharmaceutically acceptable carriers or excipients include, but are not limited to, buffers, stabilizers, surfactants, antioxidants, preservatives, sugars, polyols, amino acids, polymers.

As used herein. “buffer” refers to a buffered solution that resists changes in pH by the action of its acid-base conjugate components. The buffers of this invention have a pH in the range from about 5.0 to about 8.0; preferably from about 5.5 to about 7.0; more preferably has a pH in the range from about 5.5 to about 6.5; and most preferably has a pH of about 6.0. Examples of buffers that will control the pH in this range include histidine, acetate (e.g., sodium acetate), citrate, phosphate, succinate (such as sodium succinate), gluconate, glutamate, and other organic acid buffers. In one embodiment, a buffer suitable for use in the formulations of the invention is a histidine buffer.

The term “surfactant” generally includes those agents which protect a protein in a formulation from air/solution interface-induced stresses and solution/surface induced-stresses. For example, a surfactant may protect the protein from aggregation. Suitable surfactants may include, e.g., polysorbates, polyoxyethylene alkyl ethers such as Brij 35®, or poloxamer such as Tween 20. Tween 80, or poloxamer 188. Preferred detergents are polysorbates or Tweens, e.g., Polysorbate or Tween 20. Polysorbate 80 or Tween 80, Mirj: poloxamers, e.g., Poloxamer 188. Poloxamer 20) 407; polyoxyethylene alkyl ethers, e.g., Brij 35®. Cremophor A25. Sympatens ALM/230.

As used herein, a “polyol” is a substance with multiple hydroxyl groups, and includes sugars (reducing and nonreducing sugars), sugar alcohols and sugar acids. Non-limiting examples of polyols are sorbitol, sucrose, trehalose, glycerol, mannitol, dextrose, fructose, mannose, maltose, lactose, arabinose, xylose, ribose, rhamnose, galactose, glucose, sorbose, melezitose, raffinose, xylitol, erythritol, threitol. L-gluconate and metallic salts thereof.

Overall Response (OR) is achieved when a patient with MDS has a complete response (CR) (any type), partial remission (PR), or hematologic improvement (HI) according to the following criteria for response assessment in AML, or response assessment in MDS (see chapters below).

Overall Response will be summarized by overall response rate (ORR). 30) Complete Response (CR) is achieved when a patient has a best response of CR (any type) according to the following criteria for response assessment in AML, or response assessment in MDS (see chapters below). Complete Response for AML and MDS will be summarized by the CR rate.

OR Duration of Response—for MDS only—(OR DOR) is defined for patients who achieved CR (any type), PR, or HI as the time between the date of initial documentation of the qualifying response to the date of first documented evidence of relapse, disease progression, initiation of a new systemic anti-cancer therapy (besides HSCT), or death, whichever comes first.

CR Duration of Response—AML and MDS—(CR DOR) is defined for patients who achieved CR (any type) as the time between the date of initial documentation of the CR (or subtype) to the date of first documented evidence of non-CR response (eg, morphologic leukemia-free state [MLFS]), relapse, initiation of a new systemic anti-cancer therapy (besides HSCT), or death, whichever comes first.

OR Time to Response (MDS only): OR TTR is defined for patients who achieved CR (any type), PR, or HI as the time from the first dose of study drug to first qualifying response.

CR Time to Response (AML and MDS): CR TTR is defined for patients who achieved CR (any type) as the time from the first dose of study drug to first qualifying response.

Transfusion independence is defined as the absence of RBC and platelet transfusions for 8 weeks or longer after starting study treatment for patients with AML and 16 weeks or longer for participants with MDS. Participants with blood counts that meet the minimum parameters for use of transfusions but do not receive transfusions per investigator decision will not be counted as achieving TI. Transfusions for hemoglobin ≥9.0 g/dL or for platelets ≥50×10⁹/L will not be counted as part of the TI assessment.

RBC transfusion dependence at baseline definitions:
AML: One or more RBC transfusions given in response to hemoglobin levels of ≤9.0 g/dL within 28 days prior to the start of study treatment (FDA Guidance for Industry. Acute Myeloid Leukemia: Developing Drugs and Biological Products for Treatment. 2020).
Higher-risk types of MDS: Three or more RBC transfusions given in response to hemoglobin levels of ≤9.0 g/dL in the 16-week period prior to the start of study treatment (Platzbecker 2019). If a 16-week period is not feasible. TD will be considered for participants with ≥2 RBC transfusions in response to hemoglobin levels of ≤9.0 g/dL an 8-week period prior to study treatment initiation.

Platelet transfusion dependence for all participants is defined as receipt of any platelet transfusion within 28 days prior to the start of study treatment (FDA Guidance for Industry. Acute Myeloid Leukemia: Developing Drugs and Biological Products for Treatment. 2020).

“Subject” or “patient” includes refers to a human. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“T cell” and “T lymphocyte” are interchangeable and used synonymously herein. T cell includes thymocytes, naïve T lymphocytes, memory T cells, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD⁴⁺ T cell) CD⁴⁺ T cell, a cytotoxic T cell (CTL; CD8⁺ T cell), a tumor infiltrating cytotoxic T cell (TIL; CD8⁺ T cell), CD⁴⁺CD8⁺ T cell, a gamma-delta T cell, or any other subset of T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance. Also included are “gamma-delta T cells (γδT cells),” which refer to specialized populations of T cells possessing a distinct TCR on their surface with an ability to recognize non-classical T cell antigens, and unlike the majority of T cells in which the TCR is composed of two glycoprotein chains designated α- and β-TCR chains, the TCR in γδ T cells is made up of a γ-chain and a α-chain. Different types of γ-chains and α-chains exist, such as for example Vγ9 and Vδ2 chains which are co-expressed on Vγ9Vδ2 T cells. γδ T cells can play a role in immunosurveillance and immunoregulation, and were found to be an important source of IL-17 and to induce robust CD8+ cytotoxic T cell responses. Also included are “regulatory T cells” or “Tregs” which refer to T cells that suppress an abnormal or excessive immune response and play a role in immune tolerance. Tregs are typically transcription factor Foxp3-positive CD⁴⁺ T cells and can also include transcription factor Foxp3-negative regulatory T cells that are IL-10-producing CD⁴⁺ T cells.

As used herein, the term “unit dose” is defined as an amount of a therapeutic agent that can be administered at a given point. Dose and dosage are synonyms.

“Therapeutically effective amount” or “effective amount” used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved well-being of the patient, reduction of a tumor burden, arrested or slowed growth of a tumor, and/or absence of metastasis of cancer cells to other locations in the body.

“Treat,” “treating” or “treatment” refers to accomplishing one or more of the following responses in a patient or a cohort of patients with AML or MDS, by administering the unit dose of the CD33×Vδ2 bispecific antibody according to the different embodiments disclosed herein.

### Criteria for Response Assessment in AML (Source: Döhner 2022)

Responses are ordered from best to worst through “non-evaluable for response,” and the best applicable response should be selected for the patient:
Complete remission (CR) with (a) Bone marrow blasts <5%; (b) Absence of circulating blasts; (c) Absence of extramedullary disease; (d) Absolute neutrophil count (ANC) ≥1.0×10⁹/L (1,000/μL); (e) Platelet count ≥100×10⁹/L (100,000/μL);
CR with partial hematologic recovery (CRh) (for patients with CR, CRh, or CRi, the presence of a low percentage of circulating blasts in the blood may represent a regenerating marrow and should not be interpreted as persistent disease. In such cases the blasts generally disappear within a week) with BM, circulating blast, and EMD criteria of CR are met with BOTH:

$$ANC \geq 0.5 \times {10}^{9} / L ( 500 / μL ) AND$$

$$platelat count \geq 50 \times {10}^{9} / L ( 50 , 000 / μL ) ;$$

CR with incomplete hematologic recovery (CRi) (for patients with CR, CRh, or CRi, the presence of a low percentage of circulating blasts in the blood may represent a regenerating marrow and should not be interpreted as persistent disease. In such cases the blasts generally disappear within a week) with BM, circulating blast, and EMD CR criteria are met but with either residual neutropenia (<1.0×10⁹/L [1,000/μL]) OR thrombocytopenia (<100×10⁹/L [100,000/μL]);
Morphologic leukemia-free state (MLFS) with Both BM and circulating blast and EMD CR criteria being met but No hematologic recovery required. Note: Marrow should not merely be “aplastic”; bone marrow spicules should be present; ≥200 cells should be enumerated in the aspirate or cellularity should be at least 10% in the biopsy;
Partial remission (PR): having All hematologic criteria of CR; Decrease of bone marrow blast percentage to 5% to 25%; AND decrease of pretreatment bone marrow blast percentage by at least 50%;
No response, in those cases evaluable for response but not meeting the criteria for CR, CRh, CRi, MLFS, or PR. In addition, participants with a prior MLFS or PR who lose this response will be designated as having no response at the corresponding disease assessment;
Non-evaluable for response: this category includes patients without an adequate response evaluation (eg, death or withdrawal prior to response assessment, technically suboptimal bone marrow sample precluding assessment);
CR, CRh, or CRi without MRD (CR_{MRD-}, CRh_{MRD-} or CRi_{MRD-}): MRD testing may be completed locally and is reported separately from the response assessment in the eCRF. Response with MRD detection at low-level (CR_{MRD-}LL) is included in this category of CR, CRh or CRi without MRD. CR_{MRD-}LL is currently only defined for NPM1-mutant and CBF-AML.

Treatment failure for AML (the response landmark is defined by the response at the second post-baseline disease assessment, which is due 13 weeks (+2 weeks) after first exposure to study therapy) can be classified according to the following criteria:
Refractory disease: No CR, CRh or CRi at the response landmark. (Multiparameter flow cytometry-MRD positivity is defined as >0.1% of CD45 expressing cells with the target immunophenotype. MRD test positivity by qPCR is defined as cycling threshold (Ct)<40 and is negative if Ct ≥40 in ≥2 of 3 replicates. In NPM1-mutated and CBF-AML, CR with molecular MRD detectable at low-level (CR_{MRD-}LL) defined as <2% is designated as negative for MRD, because when measured at the end of consolidation treatment, is associated with a very low relapse rate);
Relapsed disease (after CR, CRh or CRi): Bone marrow blasts ≥5%; or reappearance of blasts in the blood in ≥2 peripheral blood samples at least one week apart; or development of extramedullary disease;
MRD relapse (after CR, CRh or CRi without MRD): Conversion from MRD negativity to MRD positivity (method independent) or Increase of MRD copy numbers ≥1 logio between any two positive samples in patients with CR_{MRD-LL}, CRh_{MRD-LL} or CRi_{MRD-LL} by qPCR (Multiparameter flow cytometry-MRD positivity is defined as >0.10% of CD45 expressing cells with the target immunophenotype. MRD test positivity by qPCR is defined as cycling threshold (Ct)<40 and is negative if Ct ≥40 in ≥2 of 3 replicates. In NPM1-mutated and CBF-AML, CR with molecular MRD detectable at low-level (CR_{MRD-}LL) defined as <2% is designated as negative for MRD, because when measured at the end of consolidation treatment, is associated with a very low relapse rate).

### Criteria for Response Assessment in MDS (Source: Zeidan 2023; Platzbecker 2019):

Responses are ordered from best to worst, with the exclusion of the hematologic improvement categories, and the best applicable response should be selected for the patient. The responses are classified as follows and must last 4 or more weeks.

### Complete Remission (CR):

Bone marrow: <5% myeloblasts; dysplasia may persist;
Peripheral blood: Hb ≥10 g/dL; platelets ≥100×10⁹/L; neutrophils ≥1.0×10⁹/L; blasts 0% (If there is a discrepancy between <5% blasts in the bone marrow with peripheral blasts >0% at time of the bone marrow evaluation, repeat the peripheral blast assessment within 2 weeks. If the peripheral blasts clear within this window, the patient will have achieved a CR).

Complete remission with limited count recovery (CRL) (CRbi—bilineage—or CRuni—unilineage):
Bone marrow: <5% myeloblasts; dysplasia may persist;
Peripheral blood: blasts 0% (if there is a discrepancy between <5% blasts in the bone marrow with peripheral blasts >0% at time of the bone marrow evaluation, repeat the peripheral blast assessment within 2 weeks. If the peripheral blasts clear within this window, the patient will have achieved a CR) and:
   CRbi: not meeting CR but only 2 of the following: Hb ≥10 g/dL; platelets ≥100×10⁹/L; neutrophils ≥1.0×10⁹/L;
   CRuni: not meeting CR but only 1 of the following: Hb ≥10 g/dL; platelets ≥100×10⁹/L; neutrophils ≥1.0×10⁹/L.
      Complete Remission with Partial Hematologic Recovery (CRh):
Bone marrow: <5% myeloblasts; dysplasia may persist;
Peripheral blood: Not meeting criteria for CR or CRL; no Hb threshold required; platelets ≥50×10⁹/L; neutrophils ≥0.5×10⁹/L; blasts 0% (If there is a discrepancy between <5% blasts in the bone marrow with peripheral blasts >0% at time of the bone marrow evaluation, repeat the peripheral blast assessment within 2 weeks. If the peripheral blasts clear within this window, the patient will have achieved a CR).

Partial remission (PR), with All CR criteria except bone marrow blasts decreased by ≥50% over pretreatment but still >5%; Cellularity and morphology not relevant.

HI-E; HI-P; HI-N: Not meeting criteria for CR or CR_{L}.

For Hematologic improvement-erythroid (HI-E) (screening period for the evaluation of transfusion burden and baseline Hb levels is ideally 16 weeks, but an 8-week screening period before treatment initiation is acceptable. For HI and TI assessments, a 16-week time window should be used. Hematologic improvements will be defined as cell lineage(s) achieving recovery at each disease assessment. If treatment interruptions/reductions following HI-E result in apparent loss of HI-E, if the response resumes with therapy re-introduction (even at a lower dose), then the transient platelet drop will not count as a loss of HI-E), the baseline transfusion burden categories include:
NTD: 0 RBCs in 16 weeks (IWG does not define the categorization of recipients of 1 or 2 RBC during the 16-week pre-treatment period, so this scenario will be considered within LTB for this study. To achieve HI-E, in addition to meeting the LTB HI-E criteria, these patients must also demonstrate a hemoglobin increase ≥1.5 g/dL);
LTB: 3-7 RBCs in 16 weeks in ≥2 transfusion episodes, ≤3 in 8 weeks (IWG does not define the categorization of recipients of 1 or 2 RBC during the 16-week pre-treatment period, so this scenario will be considered within LTB for this study. To achieve HI-E, in addition to meeting the LTB HI-E criteria, these patients must also demonstrate a hemoglobin increase ≥1.5 g/dL);
HTB: ≥8 RBCs in 16 weeks, ≥4 in 8 weeks.

For Hematologic improvement-erythroid (HI-E) confirmed based on baseline status (response durations ≥16 weeks are considered clinically meaningful):
NTD: ≥2 consecutive hemoglobin ≥1.5 g/dL ≥8 weeks in a 16-24 week period compared with the lowest mean of 2 hemoglobin measurements (omitting transfusions) within 16 weeks prior to study therapy;
LTB: 0 RBCs in ≥8 weeks within a 16-24 week period;
HTB with a Major response: 0 transfusions in ≥8 weeks within a 16-24 week period; or HTB with a Minor response: reduction by ≥50% of RBCs within ≥16 wks.

For Hematologic improvement-platelet (HI-P) (screening period for the evaluation of transfusion burden and baseline Hb levels is ideally 16 weeks, but an 8-week screening period before treatment initiation is acceptable. For HI and TI assessments, a 16-week time window should be used. Hematologic improvements will be defined as cell lineage(s) achieving recovery at each disease assessment; Dose-adjustment policy for platelet counts on treatment:
If treatment interruptions/reductions following HI-P result in apparent loss of HI-P, if the response resumes with therapy re-introduction (even at a lower dose), then the transient platelet drop will not count as a loss of HI-P.
Treatment interruptions/reductions must be accompanied by at least weekly platelet monitoring.
Two subsequent platelet counts >450×10⁹/L are sufficient to discontinue any thrombopoietin agonist therapy)
   for patients with pretreatment platelet count ≤100×10⁹/L:

$$Increase of \geq 30 \times 1 {0}^{9} / L ( starting with > 20 \times 1 {0}^{9} / L ) ; or$$

$$Increase from < 20 \times 1 {0}^{9} / L to > 20 \times 1 {0}^{9} / L AND by \geq 100 \% .$$

In addition, evolution of bleeding symptoms is to be taken into account and increments of platelets into the normal range for patients with pretreatment platelets of ≥100×10⁹/L are to be reported.

For Hematologic improvement-neutrophil (HI-N) (screening period for the evaluation of transfusion burden and baseline Hb levels is ideally 16 weeks, but an 8-week screening period before treatment initiation is acceptable. For HI and TI assessments, a 16-week time window should be used. Hematologic improvements will be defined as cell lineage(s) achieving recovery at each disease assessment),
for patients with pretreatment neutrophil count <1×10⁹/L: Increase of ≥100% and absolute increase of >0.5×10⁹/L; and
Increments of neutrophils for patients with a pretreatment ANC of ≥1.0×10⁹/L are to be reported.

No response: Not meeting criteria for CR, CRL, CRh, PR, or HI

Not evaluable: This category may include patients yet to have a response assessment, suffering early death, exiting the study early, or those with a technically suboptimal BM sample precluding assessment.

Progressive disease (PD) (patients with either PD or disease relapse, after CR, CRL, CRh, PR, or HI, are considered to have Treatment Failure at the time of either of these confirmed responses): Fulfilling any of the criteria below:
Disease progression by blasts: ≥50% relative increase in blasts and absolute increase of blast percentage by at least 5% from pretreatment sample taken before current line of therapy;
Disease progression by worsening cytopenia: new, repeated (more than once and separated by ≥7 days) need for RBC or platelet transfusions within 8 weeks, not related to acute intercurrent illness or treatment effect, in the absence of HI of at least one other blood lineage as defined above;
Progression to AML (For patients with an absolute BM blast increase to ≥20% but who have <50% relative BM blast count increase from pretreatment before current line of therapy, this could denote progression in the right clinical context where additional therapeutic options may be available with a new diagnosis of AML): ≥50% increase in blasts from baseline assessment to ≥20% blasts.

Disease relapse (after CR, CR_{L}, CRh, PR, or HI) (Patients with either PD or disease relapse (after CR, CRL, CRh, PR, or HI) are considered to have Treatment Failure at the time of either of these confirmed responses). Fulfilling any of the criteria below:
Disease relapse by blasts: absolute and relative increase in BM blasts by at least 5% and >50%, respectively, from prior assessment, or reappearance of blasts in the blood, or development of extramedullary disease (myeloid sarcoma).
Disease relapse by worsening cytopenias: decrement in one or more blood cell lineage counts by ≥50% from maximum remission/response levels for platelets or absolute neutrophil count or a reduction of Hb by 1.5 g/dL combined with an absolute reduction in the same lineage(s) as follows: Hb<10 g/dL, platelets <100×10⁹/L, or absolute neutrophils <1.0×10⁹/L or repeated (more than once and separated by ≥7 days) need for RBC or platelet transfusions which are not related to acute intercurrent illness or treatment effect; in the absence of HI of at least one other blood lineage as defined above.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

“Variant,” “.mutant” or “altered” refers to a polypeptide or a polynucleotide that differs from a reference polypeptide or a reference polynucleotide by one or more modifications, for example one or more substitutions, insertions or deletions.

The numbering of amino acid residues in the antibody constant region throughout the specification is according to the EU index as described in Kabat et al., Sequences of Proteins of Immunological Interest, 5th Edition—US Department of Health and Human Services, NIH publication no 91-3242, pp 662,680,689 (1991), unless otherwise explicitly stated.

### Sequence Listing

TABLE 1

| | | SEQ ID |
|---|---|---|
| Name | Amino acid sequence | NO: |
| HCDR1 Vδ2 (AbM) (6H4) | GRPFSNYGMG | 1 |
| HCDR2 Vδ2 (AbM) (6H4) | GISWSGGSTD | 2 |
| HCDR3 Vδ2 (AbM) (6H4) | VFSGAETAYYPSDDYDY | 3 |
| HCDR1 Vδ2 (Kabat) (6H4) | NYGMG | 4 |
| HCDR2 Vδ2 (Kabat) (6H4) | GISWSGGSTDYADSVKG | 5 |
| HCDR3 Vδ2 (Kabat) (6H4) | VFSGAETAYYPSDDYDY | 6 |
| HCDR1 Vδ2 (Chothia) (6H4) | GRPFSNY | 7 |
| HCDR2 Vδ2 (Chothia) (6H4) | SWSGGS | 8 |
| HCDR3 Vδ2 (Chothia) (6H4) | VFSGAETAYYPSDDYDY | 9 |
| HCDR1 Vδ2 (IMGT) (6H4) | GRPFSNYG | 10 |
| HCDR2 Vδ2 (IMGT) (6H4) | ISWSGGST | 11 |
| HCDR3 Vδ2 (IMGT) (6H4) | AAVFSGAETAYYPSDDYDY | 12 |
| HCDR1 Vδ2 (Contact) (6H4) | SNYGMG | 13 |
| HCDR2 Vδ2 (Contact) (6H4) | FVAGISWSGGSTD | 14 |
| HCDR3 Vδ2 (Contact) (6H4) | AAVFSGAETAYYPSDDYD | 15 |
| HCDR1 CD33 (AbM) (JL45) | GSIFSIFDMG | 16 |
| HCDR2 CD33 (AbM) (JL45) | RITNGGITN | 17 |
| HCDR3 CD33 (AbM) (JL45) | DIAANIGSNILYDDY | 18 |
| HCDR1 CD33 (Kabat) (JL45) | IFDMG | 19 |
| HCDR2 CD33 (Kabat) (JL45) | RITNGGITNYLDSVKG | 20 |
| HCDR3 CD33 (Kabat) (JL45) | DIAANIGSNILYDDY | 21 |
| HCDR1 CD33 (Chothia) (JL45) | GSIFSIF | 22 |
| HCDR2 CD33 (Chothia) (JL45) | TNGGI | 23 |
| HCDR3 CD33 (Chothia) (JL45) | DIAANIGSNILYDDY | 24 |
| HCDR1 CD33 (IMGT) (JL45) | GSIFSIFD | 25 |
| HCDR2 CD33 (IMGT) (JL45) | ITNGGIT | 26 |
| HCDR3 CD33 (IMGT) (JL45) | YADIAANIGSNILYDDY | 27 |
| HCDR1 CD33 (Contact) (JL45) | SIFDMG | 28 |
| HCDR2 CD33 (Contact) (JL45) | LVARITNGGITN | 29 |
| HCDR3 CD33 (Contact) (JL45) | YADIAANIGSNILYDD | 30 |
| VHH Vδ2 (6H4) | EVQLVESGGG LVQAGGSLRL SCAASGRPFS | 31 |
| | NYGMGWFRQA PGKKREFVAG ISWSGGSTDY | |
| | ADSVKGRFTI SRDNAKNTVY LQMNSLKPED | |
| | TAVYYCAAVF SGAETAYYPS DDYDYWGQGT | |
| | QVTVSS | |
| VHH CD33 (JL45) | QVQLVESGGG LVQAGGSLRL SCEASGSIFS | 32 |
| | IFDMGWYRRP PGAQRELVAR ITNGGITNYL | |
| | DSVKGRFSIS RDNAKNTVYL QMNSLNAEDT | |
| | AVYYCYADIA ANIGSNILYD DYWGQGTQVT VSS | |
| Heavy Chain 1 of Vδ2 arm (6H4) | EVQLVESGGG LVQAGGSLRL SCAASGRPFS | 33 |
| | NYGMGWFRQA PGKKREFVAG ISWSGGSTDY | |
| | ADSVKGRFTI SRDNAKNTVY LQMNSLKPED | |
| | TAVYYCAAVF SGAETAYYPS DDYDYWGQGT | |
| | QVTVSSEPKS SDKTHTCPPC PAPEAAGGPS | |
| | VFLFPPKPKD TLYITREPEV TCVVVSVSHE | |
| | DPEVKFNWYV DGVEVHNAKT KPREEQYNST | |
| | YRVVSVLTVL HQDWLNGKEY KCKVSNKALP | |
| | APIEKTISKA KGQPREPQVY TLPPSREEMT | |
| | KNQVSLWCLV KGFYPSDIAV EWESNGQPEN | |
| | NYKTTPPVLD SDGSFFLYSK LTVDKSRWQQ | |
| | GNVFSCSVMH EALHNHYTQK SLSLSPGK | |
| Heavy Chain 2 of CD33 arm (JL45) | QVQLVESGGG LVQAGGSLRL SCEASGSIFS | 34 |
| | IFDMGWYRRP PGAQRELVAR ITNGGITNYL | |
| | DSVKGRFSIS RDNAKNTVYL QMNSLNAEDT | |
| | AVYYCYADIA ANIGSNILYD DYWGQGTQVT | |
| | VSSEPKSSDK THTCPPCPAP EAAGGPSVFL | |
| | FPPKPKDTLY ITREPEVTCV VVSVSHEDPE | |
| | VKFNWYVDGV EVHNAKTKPR EEQYNSTYRV | |
| | VSVLTVLHQD WLNGKEYKCK VSNKALPAPI | |
| | EKTISKAKGQ PREPQVYTLP PSREEMTKNQ | |
| | VSLSCAVKGF YPSDIAVEWE SNGQPENNYK | |
| | TTPPVLDSDG SFFLVSKLTV DKSRWQQGNV | |
| | FSCSVMHEAL HNRFTQKSLS LSPGK | |
| Heavy Chain 1 of Vδ2 arm (6H4) | EVQLVESGGGLVQAGGSLRLSCAASGRPFSNYGMG | 35 |
| | WFRQAPGKKREFVAGISWSGGSTDYADSVKGRFTI | |
| | SRDNAKNTVYLQMNSLKPEDTAVYYCAAVFSGAE | |
| | TAYYPSDDYDYWGQGTQVTVSSEPKSSDKTHTCPP | |
| | CPAPEAAGGPSVFLFPPKPKDTLYITREPEVTCVVVS | |
| | VSHEDPEVKFNWYVDGVEVHNAKTKPREEQYNST | |
| | YRVVSVLTVLHQDWLNGKEYKCKVSNKALPAPIE | |
| | KTISKAKGQPREPQVYTLPPSREEMTKNQVSLWCL | |
| | VKGFYPSDIAVEWESNGQPENNYKTTPPVLDSDGSF | |
| | FLYSKLTVDKSRWQQGNVFSCSVMHEALHNRFTQ | |
| | KSLSLSPGK | |
| Heavy Chain 2 of CD33 arm (JL45) | QVQLVESGGGLVQAGGSLRLSCEASGSIFSIFDMGW | 36 |
| | YRRPPGAQRELVARITNGGITNYLDSVKGRFSISRD | |
| | NAKNTVYLQMNSLNAEDTAVYYCYADIAANIGSNI | |
| | LYDDYWGQGTQVTVSSEPKSSDKTHTCPPCPAPEA | |
| | AGGPSVFLFPPKPKDTLYITREPEVTCVVVSVSHEDP | |
| | EVKFNWYVDGVEVHNAKTKPREEQYNSTYRVVSV | |
| | LTVLHQDWLNGKEYKCKVSNKALPAPIEKTISKAK | |
| | GQPREPQVYTLPPSREEMTKNQVSLSCAVKGFYPS | |
| | DIAVEWESNGQPENNYKTTPPVLDSDGSFFLVSKLT | |
| | VDKSRWQQGNVFSCSVMHEALHNHYTQKSLSLSP | |
| | GK | |

### Further Aspects and Embodiments

As described above, in a first aspect, the disclosure relates to a unit dose of a CD33×Vδ2 bispecific antibody with a first antigen-binding region binding the Vδ2 chain of the human Vγ9Vδ2 T cell receptor, and comprising the HCDR1, HCDR2, and HCDR3, of:
a) SEQ ID NOs: 1, 2, and 3 (according to AbM), respectively;
b) SEQ ID NOs: 4, 5, and 6 (according to Kabat), respectively;
c) SEQ ID NOs: 7, 8, and 9 (according to Chothia), respectively;
d) SEQ ID NOs: 10, 11, and 12 (according to IMGT), respectively; or
e) SEQ ID NOs: 13, 14, and 15 (according to Contact), respectively; and

and a second antigen-binding region binding CD33, and comprising the HCDR1, HCDR2 and HCDR3 of:
a) SEQ ID NOs: 16, 17, and 18 (according to AbM), respectively;
b) SEQ ID NOs: 19, 20, and 21 (according to Kabat), respectively;
c) SEQ ID NOs: 22, 23, and 24 (according to Chothia), respectively;
d) SEQ ID NOs: 25, 26, and 27 (according to IMGT), respectively; or
e) SEQ ID NOs: 28, 29, and 30 (according to Contact), respectively.

The unit dose of this CD33×Vδ2 bispecific antibody is selected from the group consisting of from 385 milligrams (mg) to 0.017 mg, 385 mg, 200 mg, 110 mg, 105 mg, 100 mg, 90 mg, 80 mg, 70 mg, 60 mg, 59 mg, 58 mg, 57 mg, 56 mg, 55 mg, 54 mg, 53 mg, 52 mg, 51 mg, 50 mg, 49 mg, 48 mg, 47 mg, 46 mg, 45 mg, 44 mg, 43 mg, 42 mg, 41 mg, 40 mg, 39 mg, 38 mg, 37 mg, 36 mg, 35 mg, 34 mg, 33 mg, 32 mg, 31 mg, 30 mg, 29 mg, 28 mg, 27 mg, 26 mg, 25 mg, 24 mg, 23 mg, 22 mg, 21 mg, 20 mg, 19 mg, 18 mg, 17 mg, 16 mg, 15 mg, 14 mg, 13 mg, 12 mg, 11 mg, 10 mg, 9 mg, 8 mg, 7 mg, 6 mg, 5 mg, 4 mg, 3 mg, 2.5 mg, 2 mg, 1.7 mg, 1 mg, 0.800 mg, 0.730 mg, 0.600 mg, 0.540 mg, 0.210 mg, 0.200 mg, 0.170 mg, 0.060 mg, 0.054 mg, 0.020 mg, and 0.017 mg.

The CD33×Vδ2 bispecific antibody comprises a first antigen-binding region comprising a sequence having at least 90%, 92%, 94%, 96%, 98%, or 100% sequence identity to the single-chain moiety (VHH) of SEQ ID NO: 31; and a second antigen-binding region comprising a sequence having at least 90%, 92%, 94%, 96%, 98%, or 100% sequence identity to the single-chain moiety (VHH) of SEQ ID NO: 32. The first antigen-binding region corresponds to the Vδ2 binding arm. The second antigen-binding region corresponds to the CD33 binding arm.

The CD33×Vδ2 bispecific antibody comprises: the polypeptides set forth in SEQ ID NOs: 33 and 34. SEQ ID NO 33 corresponds to the heavy chain sequence of the Vδ2 binding arm without the RF mutation. SEQ ID NO 34 corresponds to the heavy chain sequence of the CD33 binding arm with the RF mutation.

In one embodiment, the CD33×Vδ2 bispecific antibody comprises the polypeptides set forth in SEQ ID NOs: 35 and 36. SEQ ID NO 35 corresponds to the heavy chain sequence of the Vδ2 binding arm with the RF mutation. SEQ ID NO 36 corresponds to the heavy chain sequence of the CD33 binding arm without the RF mutation.

The CD33×Vδ2 bispecific antibody of the disclosure comprises a first antigen binding region which competes for binding to human Vδ2, with an antibody having a sequence selected from SEQ ID NO: 31.

The CD33×Vδ2 bispecific antibody of the disclosure comprises a second antigen binding region which competes for binding to human CD33, with an antibody having a sequence selected from SEQ ID NO: 32.

The CD33×Vδ2 bispecific antibody of the disclosure comprises a first antigen binding region which binds the same epitope on human Vδ2 as an antibody having a sequence selected from SEQ ID NO: 31.

The CD33×Vδ2 bispecific antibody of the disclosure comprises a second antigen binding region which binds the same epitope on human CD33 as an antibody having a sequence selected from SEQ ID NO: 32.

The CD33×Vδ2 bispecific antibody and the CD33 binder are described in patent application U.S. 63/608,030. The Vδ2 arm may be prepared as described in the Examples section of patent application WO 2015/156673A1 and WO 2023/037333 A1. The art already provides the necessary teachings for the person skilled in the art to prepare the CD33×Vδ2 bispecific antibody of the disclosure, having knowledge of the actual sequences of the two arms, as provided herein.

For the generation of the CD33×Vδ2 bispecific antibody, knob-into-hole mutations are used to create heterodimers consisting of a CD33 binder fused to Fc and a Vδ2 binder fused to Fc. In these constructs, the CD33 binder is located on the Hole Fc, while the Vδ2 binder is on the Knob Fc. To achieve this, in an embodiment, the CD33 VH and human CH1 constant region are combined with a hinge on Fc, featuring several mutations: L234A/L235A/D265S_M252Y/S254T/T256E_T366S/L368A/Y407V_H435R/Y436F; and the Vδ2 binder is fused to the hinge and Knob Fc, incorporating the following mutations: C220S_L234A/L235A/D265S_M252Y/S254T/T256E_T366W.

The AAS mutations (L234A/L235A/D265S) are deliberately introduced into the Fc portion of both heavy chains to reduce the Fc receptor binding. Additionally, the YTE mutations (M252Y/S254T/T256E) are incorporated into both heavy chains of to extend the bispecific antibodies' half-life.

The RF mutations are introduced on the hole heavy chain to aid in purification. These RF mutations may be introduced on the hole heavy chain of the CD33 arm, as disclosed hereinabove or as in SEQ ID NO. 34, or on the hole heavy chain of the Vδ2 arm as in SEQ ID NO. 35. Alternatively, these RF mutations maybe introduced on both heavy chains.

The bispecific antibody CD33×Vδ2 may be produced by cultivation of recombinant Chinese hamster ovary cells, followed by isolation, chromatographic purification, and formulation.

In particular, the protein molecules—each CD33 and Vδ2 arm—may be co-produced in a CHO cell line by co-transfection of each of the encoding expression plasmids and may be purified using a two-step process involving ProA capture followed by CH1 affinity capture. Initially, the antibodies may undergo purification via a Mab Select SuRe Protein A column from GE Healthcare. The column may be prepped with PBS at pH 7.2 and then loaded with fermentation supernatant at a flow rate of 2 mL/min. Following loading, the column may undergo a wash with 4 column volumes of PBS, succeeded by elution using 30 mM sodium acetate at pH 3.5. The fractions containing protein peaks, detected by absorbance at 280 nm, may be combined and their pH neutralized to 5.0 using a 1% solution of 3 M sodium acetate at pH 9.0.

Variants of the sequences disclosed herein preferably comprise conservative modifications of the disclosed sequences. “Conservative modifications” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988)Acta Physiol Scand Suppl 643:55-67; Sasaki et al., (1988) Adv Biophys 35:1-24). Amino acid substitutions to the antibodies of the disclosure may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting variants may be tested for their characteristics using assays described herein.

The bispecific antibody (CD33×Vδ2) is a novel bispecific gd T-cell engager that recognizes the Vd2 portion of the Vγ9Vδ2 (Vgamma9 Vdelta2) T-cell receptor on a subset of T-lymphocytes and the IgC2 domain of the CD33 antigen on and malignant myeloid blasts and mature healthy myeloid cells. The antibody binding domains for both CD33 and Vδ2 are single-chain binding moieties known as variable heavy domain on a heavy chain, which contain only heavy chains; the antigen binding arms in this molecule do not contain light chains. This design is intended to enhance natural tumor recognition, engage Vγ9Vδ2 T-cells at the tumor, and selectively kill cancer cells, while avoiding the clinical risks due to widespread T-cell activation and cytotoxicity of healthy cells (as may occur with CD3-targeting therapies [Van De Vyver 2021]). This bispecific antibody also features amino acids that confer specific properties, such as abolishing interaction with Fc receptors to silence undesirable effector function, half-life extension, heterodimerization sequences known as knob in hole, and amino acid sequences (RF) on HC2 to disrupt Protein A binding pertaining to the purification strategy.

Vγ9Vδ2 T cells recognize cells that differentially express phosphoantigens (eg. malignant or stressed cells) and lead to preferential cancer cell cytotoxicity over healthy cells. Targeted recruiting the Vδ2 T-cell population is therefore hypothesized to result in a favorable safety profile with less on-target off-tumor toxicities.

### Medical Uses & Methods of Treatment

The bispecific antibody of the disclosure may be used as a medicament, in particular for use in a method of treating a patient having Relapsed or Refractory Acute Myeloid Leukemia (AML) or Myelodysplastic Neoplasms (MDS), comprising administering a therapeutically effective amount of the bispecific CD33×Vδ2 antibody of the disclosure to a patient in need thereof for a time sufficient to treat the Relapsed or Refractory AML or MDS.

The eligible patient for treatment has a diagnosis, per the WHO 2022 criteria (WHO 2022), of:
a. AML or
b. Moderate high, high, or very high risk MDS per the Molecular International Prognostic Scoring System (IPSS-M) (Bernard 2022). A Revised International Prognostic Scoring System (IPSS-R) >3.5 corresponds with risk categories of intermediate, high-risk, or very high-risk MDS (Greenberg 2012).

The new standard prognostic tool in MDS is the Molecular International Prognostic Scoring System (IPSS-M, https://mds-risk-model.com/), which classifies patients into very low (≤−1.5), low (>−1.5 to −0.5), moderate low (>−0.5 to 0), moderate high (>0 to 0.5), high (>0.5 to 1.5), and very high (>1.5) risk categories based on the percentage of bone marrow blasts, age, cytogenetics category, karyotype, number of cytopenias, and molecular data (Bernard 2022).

The IPSS-R calculator classifies MDS patients as per cytogenetic abnormalities according to the following cytogenetic prognostic subgroups:

| Cytogenetic prognostic | |
|---|---|
| subgroups | Cytogenetic abnormalities |
| Very good | −Y, del(11q) |
| Good | Normal, del(5q), del(12p), del(20q), double including del(5q) |
| Intermediate | del(7q), +8, +19, i(17q), any other single or double independent clones |
| Poor | −7, inv(3)/t(3q)/del(3q), double including −7/del(7q), Complex: 3 |
| | abnormalities |
| Very poor | Complex: >3 abnormalities |

Source: Schanz 2012
Source: https://www.mds-foundation.org/ipss-r-calculator/. Accessed 27 Oct. 2023.

Another tool in MDS, if molecular data are not available, is the revised International Prognostic Scoring System (IPSS-R), which classifies patients into very low (≤1.5), low (>1.5-3), intermediate (>3-4.5), high (>4.5-6), and very high (>6) risk categories based on the percentage of bone marrow blasts, the karyotype, and the number of cytopenias (Cheson 2006; Greenberg 2012).

IPSS-R Prognostic Score Values

| Prognostic | | | | | | | |
|---|---|---|---|---|---|---|---|
| Variable | 0 | 0.5 | 1 | 1.5 | 2 | 3 | 4 |
| Cytogenetics | Very Good | | Good | | Intermediate | Poor | Very Poor |
| BM Blast % | ≤2% | | >2%-<5% | | 5%-10% | >10% | |
| Hemoglobin | ≥10 | | 8-<10 | <8 | | | |
| Platelets | ≥100 | 50-<100 | <50 | | | | |
| ANC | ≥0.8 | <0.8 | | | | | |

Source: Greenberg 2012

Eligible patients have relapsed or refractory disease after at least one line of approved therapy. Eligible participants will have a bone marrow blast burden >5%. Patients with newly transformed secondary AML may also be eligible for treatment, provided that no standard therapeutic options exist based on the treatment prior to transformation.

The patient may have a prior or concurrent second malignancy (other than the disease under study) which natural history or treatment is unlikely to interfere with any study endpoints of safety or the efficacy of the study treatment(s).

The patient may have an ECOG performance status of 0-2 (Oken 1982).

Disease status may be evaluated according to the Response Criteria in AML (Döhner 2022) and the Response Criteria in MDS (Platzbecker 2019; Zeidan 2023).

For AML, refractory disease is diagnosed if there is no complete response (CR), no complete response with partial hematologic recovery (CRh), or no complete response with incomplete hematologic recovery (CRi) at the response landmark. The response landmark is defined by the response at the second disease assessment, which is due 13 weeks (+2 weeks) after first exposure to study therapy. If emerging preliminary efficacy data indicates that time to response may occur >13 weeks after initial study therapy exposure, the response landmark may be adjusted.

Participants with a prior CR, CRh or CRi who lose this response will be designated as having relapsed disease at the corresponding disease assessment. Participants with a prior morphologic leukemia-free state (MLFS) or partial response (PR) who lose this response will be designated as having no response at the corresponding disease assessment.

### Doses & Pharmaceutical Compositions

The present disclosure relates to unit dose of the CD33×Vδ2 bispecific antibody, wherein said unit dose is selected from the group consisting of: from 385 milligrams (mg) to 0.017 mg, 385 mg, 200 mg, 110 mg, 105 mg, 100 mg, 90 mg, 80 mg, 70 mg, 60 mg, 59 mg, 58 mg, 57 mg, 56 mg, 55 mg, 54 mg, 53 mg, 52 mg, 51 mg, 50 mg, 49 mg, 48 mg, 47 mg, 46 mg, 45 mg, 44 mg, 43 mg, 42 mg, 41 mg, 40 mg, 39 mg, 38 mg, 37 mg, 36 mg, 35 mg, 34 mg, 33 mg, 32 mg, 31 mg, 30 mg, 29 mg, 28 mg, 27 mg, 26 mg, 25 mg, 24 mg, 23 mg, 22 mg, 21 mg, 20 mg, 19 mg, 18 mg, 17 mg, 16 mg, 15 mg, 14 mg, 13 mg, 12 mg, 11 mg, 10 mg, 9 mg, 8 mg, 7 mg, 6 mg, 5 mg, 4 mg, 3 mg, 2.5 mg, 2 mg, 1.7 mg, 1 mg, 0.800 mg, 0.730 mg, 0.600 mg, 0.540 mg, 0.210 mg, 0.200 mg, 0.170 mg, 0.060 mg, 0.054 mg, 0.020 mg, and 0.017 mg; and
wherein a first antigen-binding region of the CD33×Vδ2 bispecific antibody, binds the Vδ2 chain of the human Vγ9Vδ2 T cell receptor, and comprises the HCDR1, the HCDR2, and the HCDR3, of:
a. SEQ ID NOs: 1, 2, and 3 (according to AbM), respectively;
b. SEQ ID NOs: 4, 5, and 6 (according to Kabat), respectively;
c. SEQ ID NOs: 7, 8, and 9 (according to Chothia), respectively;
d. SEQ ID NOs: 10, 11, and 12 (according to IMGT), respectively; or
e. SEQ ID NOs: 13, 14, and 15 (according to Contact), respectively; and
wherein a second antigen-binding region of the CD33×Vδ2 bispecific antibody, binds CD33 and comprises the HCDR1, HCDR2 and HCDR3 of:
a. SEQ ID NOs: 16, 17, and 18 (according to AbM), respectively;
b. SEQ ID NOs: 19, 20, and 21 (according to Kabat), respectively;
c. SEQ ID NOs: 22, 23, and 24 (according to Chothia), respectively;
d. SEQ ID NOs: 25, 26, and 27 (according to IMGT), respectively; or
e. SEQ ID NOs: 28, 29, and 30 (according to Contact), respectively.

In one embodiment, the first antigen binding region comprises a sequence having at least 90%, 92%, 94%, 96%, 98%, or 100% sequence identity to the single chain moiety (VHH) of SEQ ID NO: 31; and the second antigen binding region comprises a sequence having at least 90%, 92%, 94%, 96%, 98%, or 100% sequence identity to the single chain moiety (VHH) of SEQ ID NO: 32.

In another embodiment, the CD33×Vδ2 bispecific antibody comprises the polypeptides set forth in SEQ ID NOs: 33 and 34.

In another embodiment, the CD33×Vδ2 bispecific antibody comprises the polypeptides set forth in SEQ ID NOs: 35 and 36.

In one embodiment, the unit dose of the CD33×Vδ2 bispecific antibody is selected from 17 μg to 53.8 μg, from 20 μg to 60 μg, from 53.8 μg to 170 μg, from 60 μg to 200 μg, from 170 μg to 538 μg, from 200 μg to 600 μg, from 538 μg to 1.7 mg, from 600 μg to 2 mg, from 1.7 mg to 5.38 mg, from 2 mg to 6 mg, from 5.38 mg to 17 mg, from 6 mg to 20 mg, from 17 mg to 53.8 mg, from 20 mg to 60 mg, from 6 mg to 60 mg, from 53.8 mg to 170 mg, from 60 mg to 200 mg, from 170 mg to 538 mg, or from 200 mg to 600 mg.

In one embodiment, the unit dose in mg/Kg of the CD33×Vδ2 bispecific antibody is selected from the group consisting of from 5.35 mg/Kg to 0.00024 mg/Kg, 5.35 mg/Kg, 2.78 mg/Kg, 1.53 mg/Kg, 1.46 mg/Kg, 0.833 mg/Kg, 0.417 mg/Kg, 0.403 mg/Kg, 0.389 mg/Kg, 0.375 mg/Kg, 0.361 mg/Kg, 0.347 mg/Kg, 0.333 mg/Kg, 0.319 mg/Kg, 0.306 mg/Kg, 0.292 mg/Kg, 0.278 mg/Kg, 0.264 mg/Kg, 0.250 mg/Kg, 0.236 mg/Kg, 0.222 mg/Kg, 0.208 mg/Kg, 0.194 mg/Kg, 0.181 mg/Kg, 0.167 mg/Kg, 0.153 mg/Kg, 0.139 mg/Kg, 0.125 mg/Kg, 0.111 mg/Kg, 0.097 mg/Kg, 0.083 mg/Kg, 0.069 mg/Kg, 0.056 mg/Kg, 0.042 mg/Kg, 0.035 mg/Kg, 0.028 mg/Kg, 0.014 mg/Kg, 0.010 mg/Kg, 0.00833 mg/Kg, 0.00292 mg/Kg, 0.00278 mg/Kg, 0.00083 mg/Kg, 0.00028 mg/Kg, and 0.00024 mg/Kg.

The disclosure further relates to a pharmaceutical composition comprising the bispecific antibody as described herein, and a pharmaceutically acceptable carrier.

For therapeutic use, the bispecific CD33×62 antibodies of the disclosure may be prepared as pharmaceutical compositions containing an effective amount of the antibody as an active ingredient in a pharmaceutically acceptable carrier. The term “carrier” refers to a diluent, adjuvant, excipient, or vehicle with which the active compound is administered. Such carrier and vehicles are usually liquid. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, tonicifying agents, antioxidants, and surfactants, etc. The concentration of the antibodies of the disclosure in such pharmaceutical formulation may vary widely, i.e., formulated at 2 mg/mL, 20 mg/mL, 30 mg/mL, or at 70 mg/mL and will be selected primarily based on the required dose, fluid volumes, viscosities, etc., according to the particular mode of administration selected. The initial pharmaceutical composition having a CD33×Vδ2 antibody concentration of 2 mg/mL, 20 mg/mL, 30 mg/mL, or 70 mg/mL has as a pharmaceutically acceptable carrier: a buffer present at a concentration within the range from 10 mM to 80 mM, wherein the buffer comprises histidine, acetate, citrate, or phosphate; a tonicifying agent present at a concentration within the range from 2 to 10% (w/v), wherein the tonicifying agent is one or more sugar and/or polyol comprising sorbitol, sucrose, trehalose, glycerol, mannitol, or dextrose; a surfactant present at a concentration within the range from 0.04 to 0.2% (w/v), wherein the surfactant comprises polysorbate 20, polysorbate 80, or poloxamer 188; a stabilizer present at a concentration within the range from 20 to 120 mM, wherein the stabilizer is L-methionine; wherein the formulation has a pH within the range from 5 to 8 when in aqueous form.

In one embodiment, the pharmaceutical composition comprises the bispecific antibody, histidine, sorbitol, methionine, polysorbate 20, at a pH of 6.0. The pharmaceutical composition is a clear and colorless to light yellow solution, free of visible particulate matter. The pharmaceutical composition is preferably refrigerated between 2 to 8° C. IT can also be frozen at about −16 to 24° C. In one embodiment, the pharmaceutical formulation is a solution of the bispecific CD33 ×62 antibody in 50 mM histidine, 7.5% (w/v) sorbitol, 100 mM L-methionine, and 0.06% (w/v) Polysorbate 20 at a pH of 6.0.

The pharmaceutical composition is supplied as a sterile, preservative-free liquid that is stored at controlled temperatures and protected from light. When the pharmaceutical composition is at a concentration of 2 mg/mL, it is for example in the form of a vial of 1.35 mL. When at a concentration of 70 mg/mL, it is for example in the form of a vial of 3.53 mL.

When used for syringe IV administration, optionally with a pump, the pharmaceutical composition is diluted to the required strength using a suitable vehicle prior to use.

For lower doses, suitable vehicles include recombinant human albumin, buffer such as acetate, surfactant such as PS20, saline, and sucrose. One example of a suitable vehicle comprises 0.2 mg/n) recombinant human albumin (MA) in 10 mM acetate, 0.04% (w/v) polysorbate 20, 50 mM NaCl, 5.7% (w/v) sucrose, at a pH of 5.2.

For lower doses between about 16.5 to 804 micrograms, for the dilution to the required strength, 0.17 to 8 mL of drug product at a concentration of 2 mg/mL (“Transfer Vol” of Table 2 below) is transferred to a bottle containing 100 mL vehicle. The clinician would then take 5 to 20 mL of drug product-vehicle solution into a syringe and infuse 5 mL into the patient.

TABLE 2

| Total Dose (μg) | Transfer Vol (mL) |
|---|---|
| 16.5-17.4 | 0.17 |
| 17.5-18.4 | 0.18 |
| 18.5-19.4 | 0.19 |
| 19.5-20.4 | 0.20 |
| 20.5-21.4 | 0.21 |
| 21.5-22.4 | 0.22 |
| 22.5-23.4 | 0.23 |
| 23.5-24.4 | 0.24 |
| 24.5-25.4 | 0.25 |
| 25.5-26.4 | 0.26 |
| 26.5-27.4 | 0.27 |
| 27.5-28.4 | 0.28 |
| 28.5-29.4 | 0.29 |
| 29.5-30.4 | 0.30 |
| 30.5-31.4 | 0.31 |
| 31.5-32.4 | 0.32 |
| 32.5-33.4 | 0.33 |
| 33.5-34.4 | 0.34 |
| 34.5-35.4 | 0.35 |
| 35.5-36.4 | 0.36 |
| 36.5-37.4 | 0.37 |
| 37.5-38.4 | 0.38 |
| 38.5-39.4 | 0.39 |
| 39.5-40.4 | 0.40 |
| 40.5-41.4 | 0.41 |
| 41.5-42.4 | 0.42 |
| 42.5-43.4 | 0.43 |
| 43.5-44.4 | 0.44 |
| 44.5-45.4 | 0.45 |
| 45.5-46.4 | 0.46 |
| 46.5-47.4 | 0.47 |
| 47.5-48.4 | 0.48 |
| 48.5-49.4 | 0.49 |
| 49.5-50.4 | 0.50 |
| 50.5-51.4 | 0.51 |
| 51.5-52.4 | 0.52 |
| 52.5-53.4 | 0.53 |
| 53.5-54.4 | 0.54 |
| 54.5-55.4 | 0.55 |
| 55.5-56.4 | 0.56 |
| 56.5-57.4 | 0.57 |
| 57.5-58.4 | 0.58 |
| 58.5-59.4 | 0.59 |
| 59.5-60.4 | 0.60 |
| 60.5-61.4 | 0.61 |
| 61.5-62.4 | 0.62 |
| 62.5-63.4 | 0.63 |
| 63.5-64.4 | 0.64 |
| 64.5-65.4 | 0.65 |
| 65.5-66.4 | 0.66 |
| 66.5-67.4 | 0.67 |
| 67.5-68.4 | 0.68 |
| 68.5-69.4 | 0.69 |
| 69.5-70.4 | 0.70 |
| 70.5-71.4 | 0.71 |
| 71.5-72.4 | 0.72 |
| 72.5-73.4 | 0.73 |
| 73.5-74.4 | 0.74 |
| 74.5-75.4 | 0.75 |
| 75.5-76.4 | 0.76 |
| 76.5-77.4 | 0.77 |
| 77.5-78.4 | 0.78 |
| 78.5-79.4 | 0.79 |
| 79.5-80.4 | 0.80 |
| 80.5-81.4 | 0.81 |
| 81.5-82.4 | 0.82 |
| 82.5-83.4 | 0.83 |
| 83.5-84.4 | 0.84 |
| 84.5-85.4 | 0.85 |
| 85.5-86.4 | 0.86 |
| 86.5-87.4 | 0.87 |
| 87.5-88.4 | 0.88 |
| 88.5-89.4 | 0.89 |
| 89.5-90.4 | 0.90 |
| 90.5-91.4 | 0.91 |
| 91.5-92.4 | 0.92 |
| 92.5-93.4 | 0.93 |
| 93.5-94.4 | 0.94 |
| 94.5-95.4 | 0.95 |
| 95.5-96.4 | 0.96 |
| 96.5-97.4 | 0.97 |
| 97.5-98.4 | 0.98 |
| 98.5-99.4 | 0.99 |
| 99.5-104 | 1.0 |
| 105-114 | 1.1 |
| 115-124 | 1.2 |
| 125-134 | 1.3 |
| 135-144 | 1.4 |
| 145-154 | 1.5 |
| 155-164 | 1.6 |
| 165-174 | 1.7 |
| 175-184 | 1.8 |
| 185-194 | 1.9 |
| 195-104 | 2.0 |
| 205-214 | 2.1 |
| 215-224 | 2.2 |
| 225-234 | 2.3 |
| 235-244 | 2.4 |
| 245-254 | 2.5 |
| 255-264 | 2.6 |
| 265-274 | 2.7 |
| 275-284 | 2.8 |
| 285-294 | 2.9 |
| 295-304 | 3.0 |
| 305-314 | 3.1 |
| 315-324 | 3.2 |
| 325-334 | 3.3 |
| 335-344 | 3.4 |
| 345-354 | 3.5 |
| 355-364 | 3.6 |
| 365-374 | 3.7 |
| 375-384 | 3.8 |
| 385-394 | 3.9 |
| 395-404 | 4.0 |
| 405-414 | 4.1 |
| 415-424 | 4.2 |
| 425-434 | 4.3 |
| 435-444 | 4.4 |
| 445-454 | 4.5 |
| 455-464 | 4.6 |
| 465-474 | 4.7 |
| 475-484 | 4.8 |
| 485-494 | 4.9 |
| 495-504 | 5.0 |
| 505-514 | 5.1 |
| 515-524 | 5.2 |
| 525-534 | 5.3 |
| 535-544 | 5.4 |
| 545-554 | 5.5 |
| 555-564 | 5.6 |
| 565-574 | 5.7 |
| 575-584 | 5.8 |
| 585-594 | 5.9 |
| 595-604 | 6.0 |
| 605-614 | 6.1 |
| 615-624 | 6.2 |
| 625-634 | 6.3 |
| 635-644 | 6.4 |
| 645-654 | 6.5 |
| 655-664 | 6.6 |
| 665-674 | 6.7 |
| 675-684 | 6.8 |
| 685-694 | 6.9 |
| 695-704 | 7.0 |
| 705-714 | 7.1 |
| 715-724 | 7.2 |
| 725-734 | 7.3 |
| 735-744 | 7.4 |
| 745-754 | 7.5 |
| 755-764 | 7.6 |
| 765-774 | 7.7 |
| 775-784 | 7.8 |
| 785-794 | 7.9 |
| 795-804 | 8.0 |

For higher doses, a suitable vehicle is normal saline, i.e., a sterile solution of 0.9 grams of salt (NaCl) per 100 ml of water. As such, for higher doses, the drug product is diluted to the required strength using normal saline prior to use. Diluted pharmaceutical composition for IV administration may be administered with an IV bag.

For example, 1 mL of a vial of 1.35 mL, with an antibody concentration of 2 mg/mL is injected into an IV bag of normal saline of 50 mL, obtaining about 0.04 mg/mL strength, thereby administering 50 mL that would result in a final unit dose of about 2 mg.

So for example, 10 mL of 10 vials of 1.35 mL each, with an antibody concentration of 2 mg/mL is injected into an IV bag of normal saline of 50 mL, obtaining about 0.31 mg/mL strength, thereby administering 50 mL that would result in a final unit dose of about 20 mg.

So for example, 30 mL of 30 vials of 1.35 mL each, with an antibody concentration of 2 mg/mL is injected into an IV bag of normal saline of 50 mL, obtaining about 1.2 mg/mL strength, thereby administering 50 mL that would result in a final unit dose of about 60 mg.

Alternatively, 3 mL of a vial of 3.53 mL, with an antibody concentration of 70 mg/mL is injected into an IV bag of normal saline of 50 mL, obtaining a 4.2 mg/mL strength, thereby administering 50 mL that would result in a final unit dose of about 210 mg.

Alternatively, the pharmaceutical composition with an antibody concentration of 20 mg/mL or 30 mg/mL, each independently, is further diluted with normal saline to a unit dose between 7 mg and 70 mg.

The mode of administration for therapeutic use of the bispecific CD33×Vδ2 antibodies of the disclosure is intravenous using a formulation contained in a syringe, a syringe pump, a pump, an implanted device, osmotic pump, cartridge, micropump, an IV bag; or other means appreciated by the skilled artisan, as well known in the art.

The bispecific CD33×Vδ2 antibody of the disclosure may be administered to a patient by any suitable route, for example parentally by intravenous (IV) infusion. IV infusion can be given over as little as 15 minutes, but more often for 30 minutes, 60 minutes, 90 minutes or even 2, 3, 4, 5, or 6 hours.

Between 1 and 20 doses, (e.g., 1, 2, 3, 4, 5, 6, 7, 8, 9, 10, 11, 12, 13, 14, 15, 16, 17, 18, 19, or 20) may be administered to treat cancer, but 25, 30, 40 or more doses may be given. Administration of the bispecific CD33×Vδ2 antibody of the disclosure is repeated after two weeks.

While having described the invention in general terms, the embodiments of the invention will be further disclosed in the following examples that should not be construed as limiting the scope of the claims.

### Examples

List of Abbreviations and Definitions of Terms

| | |
|---|---|
| ADA | antidrug antibody |
| ADC | antibody-drug conjugate |
| ADR | adverse drug reaction |
| AE | adverse event |
| AESI | adverse event of special interest |
| ALP | alkaline phosphatase |
| ALT | alanine aminotransferase |
| AML | acute myeloid leukemia |
| anti-HBc | hepatitis B core antigen |
| AST | aspartate aminotransferase |
| ASTCT | American Society for Transplantation and Cellular Therapy |
| AUC | area under the serum concentration-time curve |
| AUC_{inf} | area under the concentration-time curve from time zero to infinite time |
| AUC₍₀₋ₜ₎ | area under the serum concentration versus time curve from time zero to the last |
| | measurable concentration |
| AUC_{τ} | area under the plasma concentration time curve during a dosing interval at steady-state |
| AUCₜ | area under the concentration-time curve from time zero to time t |
| AUCₗₐₛₜ | area under the serum concentration-time curve from time zero to the time of the last |
| | quantifiable concentration |
| BLI | biolayer interferometry |
| BM | bone marrow |
| BTN | butyrophilin |
| BOIN | Bayesian Optimal Interval |
| CAR | chimeric antigen receptor |
| CBC | complete blood count |
| CD | cluster of differentiation |
| CFU | colony-forming unit |
| CHO | Chinese hamster ovary |
| CI | confidence interval |
| CKD-EPI | Chronic Kidney Disease Epidemiology Collaboration |
| CL | total systemic clearance of drug after intravenous administration |
| CL/F | apparent total body clearance |
| Cₗₐₛₜ | last quantifiable concentration |
| Cₘₐₓ | maximum plasma or serum concentration |
| Cₘᵢₙ | minimum plasma or serum concentration |
| C_{trough} | trough serum concentration |
| CMP | common myeloid progenitor |
| CR | complete response |
| CRF, eCRF | case report form, electronic case report form |
| CRh | complete response with partial hematologic recovery |
| CRi | complete response with incomplete hematologic recovery |
| CR_{L} | complete response with limited count recovery |
| CRO | contract research organization |
| CRP | C-reactive protein |
| CRS | cytokine release syndrome |
| CSR | clinical study report |
| CTCAE | Common Terminology Criteria for Adverse Events |
| CYP450 | cytochrome P450 |
| DC | dendritic cell |
| DIC | disseminated intravascular coagulation |
| DLT | dose-limiting toxicity |
| DOR | duration of response |
| DPBS | Dulbecco's phosphate buffered saline |
| EC₅₀ | 50% effective concentration |
| ECD | extracellular domain |
| ECG | electrocardiogram |
| ECOG | Eastern Cooperative Oncology Group |
| eDC | electronic data capture |
| ELN | European Leukemia Network |
| EMA | European Medicines Agency |
| EMD | extramedullary disease |
| EOI/EOF | end of infusion/end of flush |
| EOT | end of treatment |
| EEA | European Economic Area |
| ELISA | enzyme-linked immunosorbent assay |
| E:T | effector to target |
| EU | European Union |
| FasL | Fas ligand |
| Fc | fragment crystallizable |
| FIH | first in human |
| FOIA | Freedom of Information Act |
| FPPE | formalin-fixed paraffin |
| gd | gamma-delta |
| GCP | Good Clinical Practice |
| G-CSF | granulocyte colony-stimulating factor |
| GLP | Good Laboratory Practice |
| GMP | granulocyte-monocyte progenitor |
| HAART | highly active antiretroviral therapy |
| HBV | hepatitis B virus |
| HBsAg | hepatitis B surface antigen |
| HC | heavy chain |
| HI | hematologic improvement |
| HIV | human immunodeficiency virus |
| HLH | hemophagocytic lymphohistiocytosis |
| HLH/MAS | hemophagocytic lymphohistiocytosis/macrophage activation syndrome |
| HSC | hematopoietic stem cell |
| HSCT | hematopoietic stem cell transplant |
| HSPC | hematopoietic stem and progenitor cells |
| HTD | high transfusion burden |
| IB | Investigator's Brochure |
| ICANS | mmune effector cell associated neurotoxicity syndrome |
| ICE | immune effector cell-associated encephalopathy |
| ICF | informed consent form |
| ICH | International Council for Harmonisation |
| IEC | Independent Ethics Committee |
| IFN | interferon |
| IgV | immunoglobulin-like domain |
| Ig | immunoglobulin |
| IgC2 | C2-set immunoglobulin-like domain |
| IHC | immunohistochemistry |
| ILS | increased life span |
| IMP | investigational medicinal product |
| INR | international normalized ratio |
| IRB | Institutional Review Board |
| IRR | infusion-related reaction |
| IPPI | Investigational Product Preparation Instructions |
| IPSS-M | Molecular International Prognostic Scoring System |
| IPSS-R | Revised International Prognostic Scoring System |
| IRR | infusion-related reaction |
| ISH | in situ hybridization |
| ITIM | immunoreceptor tyrosine-based inhibitory motif |
| IV | intravenous |
| IWG | International Working Group |
| IWRS | interactive web response system |
| JEISR | Janssen Electronic Inbound Safety Reporting |
| J&J BodyMap | Johnson & Johnson Human Tissue BodyMap |
| kDa | kilodalton |
| KO | knock out |
| LAR | legally authorized representative |
| LDH | lactate dehydrogenase |
| LFT | liver function test |
| LSC | leukemic stem cell |
| LTB | low transfusion burden |
| MABEL | minimal anticipated biological effect level |
| MAS | macrophage activation syndrome |
| MDS | myelodysplastic neoplasms |
| MDSC | myeloid-derived suppressor cells |
| MedDRA | Medical Dictionary for Regulatory Activities |
| MEP | megakaryocytic-erythroid progenitor |
| MHC | major histocompatibility complex |
| MLFS | morphologic leukemia-free state |
| MoA | mechanism of action |
| MPP | multipotent progenitor |
| MRD | measurable residual disease |
| MSD | Meso Scale Discovery |
| MTD | maximum tolerated dose |
| n | number |
| N | number (total sample size) |
| NC | not calculated |
| NCI | National Cancer Institute |
| NGS | next generation sequencing |
| NK | natural killer |
| NS | normal saline |
| NTD | nontransfused |
| OR | overall response |
| ORR | overall response rate |
| OS | overall survival |
| pAg | phosphoantigen |
| PBMC | peripheral blood mononuclear cell |
| PBS | phosphate buffered saline |
| PK | pharmacokinetics |
| PR | partial response |
| PS20 | polysorbate 20 |
| PSMA | prostate-specific membrane antigen |
| Q | blood flow |
| QTcF | QTc according to Fridericia's formula |
| QXW | every X weeks where X is defined as the number of weeks |
| RO | receptor occupancy |
| R/R | relapsed or refractory |
| RP2D | recommended phase 2 dose |
| RT-PCR | real-time polymerase chain reaction |
| SAE | serious adverse event |
| SAR | serious adverse reaction |
| sCD33 | soluble CD33 |
| SD | standard deviation |
| SE | standard error |
| SEM | standard error of the mean |
| SNP | single nucleotide polymorphisms |
| SOS | sinusoidal obstruction syndrome |
| SPR | surface plasmon resonance |
| SUSAR | suspected unexpected serious adverse reaction |
| t_{1/2} | half-life |
| TAA | tumor-associated antigen |
| Tbili | total bilirubin |
| TCR | T cell receptor |
| TEAE | treatment-emergent adverse event |
| TI | transfusion independence |
| TIL | tumor-infiltrating lymphocyte |
| TGI | tumor growth inhibition |
| TLS | tumor lysis syndrome |
| T_{1/2} | terminal half-life |
| Tₗₐₛₜ | time of the last quantifiable serum concentration |
| Tₘₐₓ | time to reach maximum serum concentration |
| TNF-α | tumor necrosis factor - alpha |
| TRAIL | tumor-necrosis-factor-related apoptosis-inducing ligand |
| TTR | time to response |
| UK | United Kingdom |
| TMDD | target-mediated drug disposition |
| ULN | upper limit of normal |
| USA | United States of America |
| USPI | United States Prescribing Information |
| V/F | apparent volume of distribution |
| V_{z} | volume of distribution in the terminal phase |
| V_{c} | volume of the central compartment |
| Vₚ | volume of the peripheral compartment |
| vs | versus |
| VHH | variable heavy domain on a heavy chain |
| WBC | white blood cell |
| WHO | World Health Organization |

The bispecific antibody CD33×Vδ2 with the sequences of SEQ ID NO. 33 and 34, is the subject of the following experiments and studies, collected in Examples 1-5. This antibody has a molecular mass of 81.589 kDa and isoelectric points ranging from 6.0-6.8. The absorptivity constant at 280 nM was determined to be 1.54 (mg/mL)⁻¹ cm⁻¹.

### Example 1. Pharmacokinetics and Metabolism in Animals

To support FIH dose projection, the PK of the CD33×Vδ2 bispecific antibody was investigated in a non-GLP study after IV dosing in female cynomolgus monkeys. The study incorporated a limited panel of safety endpoints.

### Methods of Analysis

A fit-for-purpose electrochemiluminescence immunoassay method was qualified utilizing a Fab reagent derived from a mouse monoclonal anti-human IgG antibody as capture and goat polyclonal anti-human IgG detection reagent, to quantify serum concentrations of the CD33×Vδ2 bispecific antibody in a non-GLP cynomolgus monkey study where the lower limit of quantification was 0.01000 μg/mL.

### Absorption and Pharmacokinetics

Serum PK parameters of the CD33×Vδ2 bispecific antibody were characterized in female cynomolgus monkeys (3/group) following IV administration at 0.05, 0.2, and 1 mg/kg in a non-GLP study (Table 3). IV doses of the antibody were administered as a solution at 1 mL/kg in a vehicle consisting of 50 mM histidine, 7.5% sorbitol, 100 mM methionine, and 0.06% PS20 at pH 6.0. Vehicle control was administered on Days 1 and 22 to Group 1 animals. The CD33×Vδ2 bispecific antibody was administered at 0.05 mg/kg on Day 1 and 0.2 mg/kg on Day 22 to Group 2 animals, and at 1 mg/kg on Day 1 to Group 3 animals. Blood samples for serum bioanalysis were collected for up to 6 weeks from Group 1 and 2 animals and up to 30 days from Group 3 animals.

Mean antibody exposure (Cₘₐₓ and AUCₗₐₛₜ) increased in an approximate dose-proportional manner between 0.05 and 1 mg/kg. For Group 2, mean Cl, V_{z}, T_{1/2} values were calculated following the 0.05 mg/kg dose on Day 1 (8.28 mL/day/kg, 63.3 mL/kg, and 5.31 days, respectively) but not at 0.2 mg/kg dose administered on Day 22 in the same animals due to loss of exposure, which is likely due to ADA (formal ADA analysis not performed). For Group 3, following the 1 mg/kg dose, only limited PK parameters were calculated due to limited confidence on the clearance parameters because AUC % extrapolated values were >20% for all the animals.

TABLE 1
Mean (±SD) Serum CD33xVδ2 bispecific antibody PK Parameters Following IV Administration in Female Cynomolgus Monkeys

| | | Dose | | Cₘₐₓ | Tₘₐₓ^{b} | AUCₗₐₛₜ | Tₗₐₛₜ^{b} | AUC_{inf} | CL | V_{z} | T_{1/2} |
|---|---|---|---|---|---|---|---|---|---|---|---|
| Group | Day | (mg/kg) | N | (μg/mL) | (day) | (μg · day/mL) | (day) | (μg · day/mL) | (mL/day/kg) | (mL/kg) | (day) |
| 2 | 1 | 0.05 | 3 | 1.61 ± 0.316 | 0.02 | 5.01 ± 1.06 | 16 | 6.07 ± NC^{c} | 8.28 ± NC^{c} | 63.3 ± NC^{c} | 5.31 ± NC^{c} |
| | | | | | (0.02-0.02) | | (7-21) | | | | |
| | 22 | 0.2 | 2^{a} | 7.20 ± NC | 0.02 | 7.67 ± NC | 2 | NC ± NC^{d} | NC ± NC^{d} | NC ± NC^{d} | NC ± NC^{d} |
| | | | | | (0.02-0.02) | | (2-2) | | | | |
| 3 | 1 | 1 | 3 | 34.9 ± 3.05 | 0.02 | 97.7 ± 8.32 | 10 | NC ± NC^{e} | NC ± NC^{e} | NC ± NC^{e} | NC ± NC^{e} |
| | | | | | (0.02-0.02) | | (10-10) | | | | |

Serum samples for bioanalysis were obtained from blood collected prior to each dose and at timepoints up to 456 hours (19 days) after the 0.05 mg/kg dose, up to 480 hours (42 days which is relative to the dosing on Day 1 with 0.05 mg/kg) after the 0.2 mg/kg dose, and up to 720 hours (30 days) after the 1 mg/kg dose. Noncompartmental analysis was performed to derive the PK parameters.
^{a}PK parameters were not determined for 1 animal with only a single quantifiable CD33xVδ2 bispecific antibody concentration.
^{b}Median [range]
^{c}For 1 animal, the AUC % obtained by extrapolation was >20% and excluded from statistics.
^{d}AUC % obtained by extrapolation was >20%
^{e}For all animals, the AUC % obtained by extrapolation was >20% (excluded from statistics) following exclusion of Day 14 PK data (Tlast in Group 3 animals) from NCA analysis which are suspected to be impacted by ADA-mediated accelerated clearance based on the sudden loss in the antibody serum exposure (ADA analysis not performed in this study).
Abbreviations: See List of Abbreviations and Definitions of Terms.

### Example 2. Estimation of Clinical Doses Based on Nonclinical Data

The proposed FIH starting dose based on available data is 0.017 mg by IV administration. The starting dose for the CD33×Vδ2 bispecific antibody was projected using a MABEL-based approach matching the median EC₅₀ of cytokine release (IFN-γ) obtained from an in vitro γδ T-cell cytotoxicity assay in whole blood co-cultured with Kasumi-1 cancer cells to the projected serum Cₘₐₓ, while using the projected human PK parameters which were allometrically scaled from in vivo cynomolgus monkey PK data. The MABEL assay conditions were relevant to patients with AML. The MABEL-based approach is consistent with the recommendations by European Medicines Agency and retrospective analysis performed by members of the FDA's CDER Office of Hematology and Oncology Products (EMA 2017, Saber 2017).

The MABEL assay was performed using whole blood from 6 to 7 healthy human donors. Cancer cells, i.e. Kasumi-1 cells, were added to obtain an E:T ratio of 1:1 based on pan T-cells (relative E:T ratio is 1:37 with Vδ2 T-cells as effector cells based on the median range of 2.67% Vδ2 T-cells across 6 to 7 healthy donors). The in vitro E:T ratio is relevant and representative of E:T in AML patients based on published data (Krupka 2014). The negligible and variable cytotoxicity observed in the MABEL assay could not be modeled to determine the EC₅₀ values, thus precluding use of cytotoxicity data to derive the FIH starting dose. Thus, model-estimated EC₅₀ values of early T-cell activation (48 hours) on pan T-cells (CD25⁺CD3⁺) and γδ T-cells (CD25⁺γδ⁺), late T-cell activation (96 hours) on pan T-cells (CD25⁺CD3⁺) and γδ T-cells (CD25⁺γδ⁺) as well as cytokine release at 48 and 96 hours from the MABEL assay were evaluated to calculate the starting dose. Maximum γδ T-cell activation was ≤20% (ranging between 11% and 20% at both 48 and 96 hours) with negligible (6% to 7%) pan T-cell activation at both timepoints. Since only ≤20% γδ T-cells were activated, which represents a small subpopulation of pan T-cells (˜2.67%), in addition to negligible pan T-cell activation, a starting dose based on these parameters would be overly conservative. IFN-γ is released upon the activation of γδ T-cells and absence of this release is associated with inhibitory T-cell activities (associated with IL-17 production by these cells), thus emphasizing its relevance to the biology of γδ T-cells (Rao 2024, Park 2021). Based on the totality of the data, a starting dose based on IFN-γ release (EC₅₀=0.1 nM) at 48 hours can be considered safe and relevant (based on the biology of Vδ2 T-cells).

To estimate human PK parameters, based on the observed dose linearity in cynomolgus monkeys (weighing on average 2.9 kg), clearance and volume of distribution for cynomolgus monkeys were calculated using a two-compartment model fit and were allometrically scaled (assuming an average body weight of 72 kg for patients). Exponents for allometric scaling were 0.85 for clearance associated parameters (CL and Q₁₂) and 1.0 for distribution volume associated parameters (V_{C} and V_{P}). Although human PK for other antibodies with half-life extending mutations (primarily for viral targets) have been reported to be better projected using other allometry coefficients (Haraya 2023), traditional mAb allometric scaling parameters (Deng 2011) are assumed due to uncertainty on (1) half-life extending mutation impacts for membrane-bound targets (existing literature focuses on viral targets; Haraya 2023), and (2) the novel combination of mutations and components in CD33×Vδ2 bispecific antibody (bispecific VHH with half-life extending mutation and Fc silencing). Cynomolgus monkeys showed dose linear PK and the γδ arm was not cross-reactive in cynomolgus monkeys; TMDD could still be possible in human and will have to be determined in the clinic.

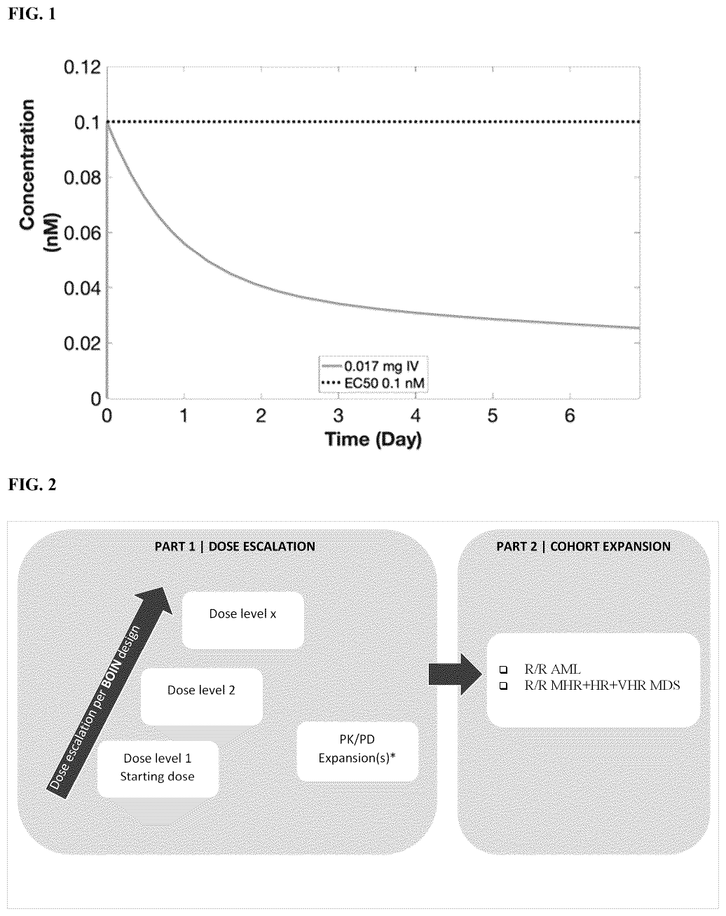

Based on the above considerations, the FIH starting dose for the CD33×Vδ2 bispecific antibody was determined to be 0.017 mg via IV administration. As illustrated in FIG. 1, the proposed starting dose is projected to have the mean Cₘₐₓ equal to the targeted MABEL concentration.

### Example 3. CD33×Vδ2 Bispecific Antibody, 2 mg/mL, Drug Product (DP)

The general information on the DP are summarized in Table 4.

TABLE 4
General Information for the Drug Product

| Drug Product | CD33 × Vδ2 bispecific antibody | |
|---|---|---|
| Target | CD33 and Vγ9δ2 receptors | |
| Formulation | 2 mg/mL of antibody | |
| | In 50 mM histidine, 7.5% (w/v) sorbitol, 14.92 mg/mL | |
| | methionine | |
| | with 0.06% (w/v) Polysorbate 20 at pH 6.0 | |
| Osmolality (Target) | 623 | mOsm/kg |
| Nominal Filling Volume | 1.00 | mL |
| Strength | 2 | mg/vial |
| Recommended Storage Conditions | 5 ± 3° C., protected from light | |
| Flat or Variable Dosing | Flat Dosing | |
| Proposed Maximum Flat Dose (mg) | 16.74 | mg* |
| Highest Dose Level (mg/kg) | 0.279 | mg/kg |
| Average Body Weight (kg): | 60 | kg* |
| Administration Time (hour): | 1 | hour(s)** |

| | |
|---|---|
| Administration route | Intravenous |

*Used for calculating Endotoxin acceptance criterion
**Administration time is less than or equal to 1 (hour).

### Example 4. CD33×Vδ2 Bispecific Antibody, 70 mg/mL, Drug Product (DP)

The general information on the DP are summarized in Table 5.

TABLE 5
General Information for the Drug Product

| Drug Product | CD33 × Vδ2 bispecific antibody | |
|---|---|---|
| Target | CD33 and Vγ9δ2 receptors | |
| Formulation | 70 mg/mL of antibody | |
| | In 50 mM histidine, 7.5% (w/v) sorbitol, 14.92 mg/mL | |
| | methionine with 0.06% (w/v) Polysorbate 20 at pH 6.0 | |
| Nominal Volume | 3.0 | mL |
| Strength | 210 | mg/vial |
| Recommended Storage Conditions | 2-8° C., protected from light | |
| Flat or Variable dosing | Flat | |
| Proposed Maximum Dose (mgs) | 585.9 | mg |
| Average Body Weight (kg): | 60 | kg* |
| Administration Time (hour): | 1 | hour(s)** |

| | |
|---|---|
| Administration route | Intravenous |

*Used for calculating Endotoxin acceptance criterion
**Administration time is less than or equal to 1 (hour).

### Example 5. Clinical Protocol: a Phase 1, First-In-Human, Dose Escalation Study of a CD33×Vδ2 Bispecific Antibody for Relapsed or Refractory Acute Myeloid Leukemia or Myelodysplastic Neoplasms

### Protocol Summary

### Synopsis

The CD33×Vδ2 bispecific antibody is a novel bispecific gamma-delta (γδ) T-cell engager intended to enhance natural tumor recognition, engage Vγ9Vδ2 T-cells at the tumor, and selectively kill cancer cells, while avoiding the clinical risks due to widespread T-cell activation and cytotoxicity of healthy cells.

### Objectives and Endpoints

The primary objectives of the study are to determine the safety and tolerability of the CD33×Vδ2 bispecific antibody (Part 1 and Part 2) and to identify the recommended Phase 2 dose(s) (RP2D[s]) in participants with relapsed or refractory (R/R) acute myeloid leukemia (AML) or R/R higher-risk types of myelodysplastic neoplasms (MDS). Secondary objectives are to assess the pharmacokinetics (PK), immunogenicity and to evaluate the preliminary clinical activity of the CD33×Vδ2 bispecific antibody (Part 2) at the RP2D(s).

### Hypothesis

No formal statistical hypothesis testing will be conducted in this study. The study will evaluate the following:

Dose escalation (Part 1): The RP2D(s) and schedule(s) of the CD33×Vδ2 bispecific antibody may be identified such that the estimate of the dose-limiting toxicity (DLT) rate is below or equal to the target rate of 28%.

Cohort expansion (Part 2): the CD33×Vδ2 bispecific antibody is safe and shows clinical activity at the RP2D(s).

### Overall Design

This is a first-in-human, open-label, multicenter, Phase 1 study to evaluate the safety, PK, pharmacodynamics, immunogenicity, and preliminary clinical activity of the CD33×Vδ2 bispecific antibody monotherapy administered in participants with R/R AML or R/R higher-risk types of MDS. Evaluations may also be conducted to assess if malignancies with specific mutational/immunologic signatures respond to treatment with the CD33×Vδ2 bispecific antibody.

The study will be conducted in 2 parts: Part 1 (Dose Escalation) and Part 2 (Cohort Expansion).

Part 1 is designed to determine the RP2D(s) and dosing regimen(s) of the CD33×Vδ2 bispecific antibody based on safety, PK, pharmacodynamics, and preliminary clinical activity. PK/pharmacodynamic expansion cohort(s) with ˜10 participants will be used to further delineate the PK parameters of the CD33×Vδ2 bispecific antibody at a dose regimen associated with a favorable safety profile and initial efficacy. Part 2 is designed to further assess safety and efficacy of the RP2D(s) in participants with specific subtypes of R/R myeloid malignancies. During the study, safety will be monitored at each dose escalation in Part 1 and at regular intervals during cohort expansion in Part 2. Decisions will be based on the review of all available data including, but not limited to PK, pharmacodynamic, safety, and preliminary clinical activity.

The end of study (study completion) is defined as last study assessment for the last participant in the study.

### Number of Participants

A target of approximately 100 participants will be enrolled in this study. The actual number of participants in Part 1 will depend on the number of dose levels explored and the number of participants enrolled at each dose level and route of administration. The actual number of participants in Part 2 will depend on the number of RP2Ds selected with each RP2D being tested in approximately 20 participants per disease group.

### Treatment Groups and Duration

Participants will receive the CD33×Vδ2 bispecific antibody as a monotherapy, administered intravenously, until progression, relapse, refractory response, intolerable toxicity, withdrawal of consent, or the investigator determines that it is in the best interest of the participant to discontinue study drug treatment. Ongoing therapy may be permitted for participants continuing to derive clinical benefit.

### Efficacy Evaluations

Response assessments according to corresponding AML and MDS response criteria will be performed.

### Pharmacokinetic, Receptor Occupancy, Immunogenicity Evaluations and Biomarker Evaluations

Whole blood, serum, and bone marrow samples will be assessed to evaluate PK and pharmacodynamics of the CD33×Vδ2 bispecific antibody; samples may also be used for genetic/gene expression/proteomic assays to identify potential biomarkers associated with response, resistance, or mechanism.

### Safety Evaluations

Safety of the CD33×Vδ2 bispecific antibody will be assessed by medical history, adverse event (AEs) monitoring, clinical laboratory test results, electrocardiograms, vital sign measurements, physical examination findings (including basic neurologic exam), and Eastern Cooperative Oncology Group performance status score.

### Statistical Methods

No formal statistical hypothesis testing will be conducted in this study. Dose escalation or de-escalation decisions will be guided by the Bayesian Optimal Interval Design with the intention to select an RP2D such that the estimate of the DLT rate is below or equal to the target rate of 28%.

### Schema

A diagram of the study design, including the dose escalation plan, is provided in FIG. 2. The figure provides an example of the planned dose escalation scheme.

Note that at least one RP2D could be explored in Part 2. Part 2 cohorts may be revised as supported by emerging data.

### Introduction

The CD33×Vδ2 antibody is a novel bispecific gd T-cell engager that recognizes the Vd2 portion of the Vγ9Vδ2 (Vgamma9 Vdelta2) T-cell receptor on a subset of T-lymphocytes and the CD33 antigen on and malignant myeloid blasts and mature healthy myeloid cells. This design is intended to enhance natural tumor recognition, engage Vγ9Vδ2 T-cells at the tumor, and selectively kill cancer cells, while avoiding the clinical risks due to widespread T-cell activation and cytotoxicity of healthy cells (as may occur with CD3-targeting therapies (Van De Vyver 2021). The CD33×Vδ2 bispecific antibody is being developed for the treatment of AML or higher-risk types of MDS, which no longer responds to standard therapies.

A summary of the in vitro and in vivo pharmacology, safety pharmacology, and toxicology are presented within this section.

### Study Rationale

Targeted immunotherapy in AML and higher-risk MDS remains a substantial clinical challenge due to the heterogenous nature of the myeloblasts and the lack of disease-specific antigens. While several immunotherapies targeting CD33, including CAR-T and CAR-NK cell therapies, are being evaluated clinically, challenges have been observed with CD3 T-cell engager therapies targeting CD33 due to an insufficient benefit-risk profile; specifically, compounds AMG 673, AMG 330, and JNJ-67571244 stopped enrollment prior to determining the RP2D regimen (Amgen 2020; Amgen 2022; Narayan 2024). CD33×Vδ2 T-cell engagers represent a potentially safer and novel treatment that may address unmet medical needs of patients with AML or higher-risk types of MDS whose disease no longer responds to standard therapies. Bispecific Vδ2 T-cell engagers could potentially provide preferential cytotoxicity against cancer over healthy target-expressing cells. In addition, activation of a small T-cell subset would avoid pan T-cell activation that is known to cause CRS.

The antibody is a bispecific CD33×Vδ2 antibody developed to bind to CD33 as TAA on AML and MDS blast cells and the Vδ2 chain on Vγ9Vδ2 T cells to provide potent antitumor efficacy with potentially minimal toxicity. Vγ9Vδ2 T cells recognize cells that differentially express phosphoantigens (i.e., malignant or stressed cells) and lead to preferential cancer cell cytotoxicity over healthy cells. We hypothesize that recruiting the Vδ2 T cell population may result in a favorable safety profile with less on-target off-tumor toxicities.

### AML

AML is an aggressive, heterogeneous clonal disease of the blood and BM. Disease remission can be achieved with standard induction chemotherapy, but R/R disease remains a challenge due to persistence of leukemic stem cells and represents an unmet medical need. To date, immunotherapy in AML has not been successful and has shown limited clinical activity with substantial toxicities owing to the lack of suitable target antigens.

### Higher-Risk Types of MDS

MDS represents a heterogeneous group of malignant hematopoietic stem cell disorders that are characterized by cytopenias, myeloid dysplasia, and a risk of transformation to AML (Cheson 2006). As with AML, HSCT represents the only potentially curative option with limited availability (Prebet 2016). Currently there are no approved treatment options for patients with relapsed/refractory disease. As in AML, immunotherapy in higher-risk types of MDS has not been successful and has been associated with substantial toxicity.

γδ

The unique biology and distinctive features of Vδ2 T cells make them an attractive immune subset to explore for cancer immunotherapy. γδ T-cell based therapeutic strategies currently in the clinic include allogeneic γδ T-cell transfer (ex vivo expanded γδ T-cell infusion), CAR γδ T cells, γδ TCR-T therapy, agonist antibodies (anti-BTN3A antibody), and Vδ2-engaging antibodies.

### CD33

CD33 is a clinically validated target for AML.

### Background

### Summary of Nonclinical Safety Assessment, Nonclinical Pharmacokinetics, and Immunogenicity

γδ T-cell populations are divergent in TCR chain usage, frequency, distribution, and functional phenotype between rodents and primates, and therefore rodents were not considered a pharmacologically relevant species for safety testing. The CD33 binder of the CD33×Vδ2 bispecific antibody was cross-reactive to cynomolgus monkey CD33; however, the Vδ2 binder did not cross-react with monkey Vδ2. Therefore, a pharmacologically relevant preclinical species for toxicity testing could not be identified, and the nonclinical safety strategy leverages relevant literature, and an in silico, in vitro, ex vivo approach.

### Nonclinical Safety Assessment

In vivo models were not considered relevant for safety testing because of a lack of a cross-reactive preclinical species.

### Pharmacokinetics and Immunogenicity Profile in Cynomolgus Monkeys

In a single IV administration PK cynomolgus monkey study of the CD33×Vδ2 bispecific antibody, mean serum exposure (Cₘₐₓ and AUCₗₐₛₜ) increased in an approximate dose-proportional manner.

### Clinical Studies

This will be the first administration of the CD33×Vδ2 bispecific antibody in humans; therefore, no clinical experience is available.

While there is limited safety information from clinical-stage Vδ2 T-cell-engaging molecules such as LAVA-051 (CD1dxVδ2) and LAVA-1207 (PSMAxVδ2) in the public domain (Kater 2022; Lameris 2021; Lameris 2022), the available data indicate that AEs most frequently observed were low grade CRS and infusion-related reactions; such effects are expected to be manageable and monitorable clinically.

### Benefit-Risk Assessment

This is the first clinical study of the CD33×Vδ2 bispecific antibody. The risks and benefits associated with the administration of the CD33×Vδ2 bispecific antibody in this FIH study are unknown. In terms of efficacy, it is possible that the participant's disease does not respond to the study drug or that the participant may receive a subtherapeutic dose, particularly in the lower dose cohorts. While T-cell engagers have been tested in AML and MDS, no such therapies have been approved for either indication. At the same time, other malignancies, including certain acute lymphoblastic leukemia, lymphoma, multiple myeloma, non-small cell lung cancer, and melanoma indications, have shown beneficial responses to T-cell engagers that have led to several approvals and these positive results suggest a potential therapeutic opportunity for AML/MDS.

A pharmacologically relevant (cross-reactive) nonclinical animal species was not identified for the CD33×Vδ2 bispecific antibody, therefore the available information regarding potential toxicities associated with the antibody administration in humans is based on: (1) safety data from nonclinical and clinical studies with T-cell immunotherapies (eg, T-cell activation and tumor cell lysis); (2) the CD33-targeted mechanism of action (though sparing of nonmalignant CD33+ cell populations is anticipated); and (3) the intravenous route of administration. Administration of the CD33×Vδ2 bispecific antibody may involve risks that are currently unforeseen. Taking into account the measures taken to minimize risk to participants in this study, the potential risks identified in association with the CD33×Vδ2 bispecific antibody are justified by the anticipated benefits that may be afforded to participants with relapsed or refractory (R/R) AML or R/R higher-risk types of MDS.

### Objectives and Endpoints

| | |
|---|---|
| Objectives | Endpoints |

Primary

| | |
|---|---|
| Part 1 (Dose Escalation) & Part 2 (Dose | Incidence, type and severity of adverse events, |
| Expansion): Characterize safety across tested | including DLT(s) |
| dose regimens and define the RP2D(s) of the | |
| CD33 × Vδ2 bispecific antibody | |

Secondary

| | |
|---|---|
| To assess the PK and immunogenicity of the | Serum concentration-time profiles and PK |
| CD33 × Vδ2 bispecific antibody | parameters for the CD33 × Vδ2 bispecific |
| | antibody including but not limited to AUC_{τ}, |
| | Cₘₐₓ, and C_{trough} |
| | Presence of anti-drug antibodies of the CD33 × Vδ2 |
| | bispecific antibody. |
| To evaluate the preliminary clinical activity of the | Complete Response (CR/CRh/CRi/) in AML per |
| CD33 × Vδ2 bispecific antibody in participants with | ELN 2022 (Döhner 2022) |
| relapsed/refractory AML or relapsed/refractory | Overall Response (CR/CR_{L}/CRh/PR/HI) and |
| higher-risk types of MDS | Complete Response (CR/CR_{L}/CRh) in MDS per |
| | IWG 2023 (Zeidan 2023; Platzbecker 2019) |
| | Duration of response |
| | Time to response |
| | Transfusion independence |

Exploratory
Explore biomarkers of response to the CD33 × Vδ2 bispecific antibody which may include mutation and
expression profiles, T-cell frequency, activation, expansion, and characterization,
cytokine/chemokine profile, and immune cell subsets associated with response/resistance.
Explore the relationships between PK, pharmacodynamics, biomarkers, AE profiles, and clinical activity
or resistance to the CD33 × Vδ2 bispecific antibody
To evaluate RO of Vδ2 and CD33

### Hypothesis

No formal statistical hypothesis testing will be conducted in this study. The study will evaluate:

Dose Escalation (Part 1): the RP2D regimen(s) of the CD33×Vδ2 bispecific antibody may be identifined such that the estimate of the DLT rate is below or equal to the target rate of 28%.

Cohort Expansion (Part 2): the CD33×Vδ2 bispecific antibody is safe with clinical activity at the RP2D regimen(s).

### Study Design

### Overall Design

This is a FIH, open-label, Phase 1 study to evaluate the safety, PK, pharmacodynamics, and preliminary clinical activity of the CD33×Vδ2 bispecific antibody monotherapy administered to adult participants with relapsed or refractory myeloid malignancies that include AML or higher-risk types of MDS. The overall goal of the study is to determine the dose and regimen that may be used in future clinical development. Evaluations may also be conducted to assess if malignancies with specific mutational/immunologic signatures respond to treatment with the antibody. In addition, participant demographic details such as race and ethnicity will be collected to ensure an adequately broad study population that is representative of the broader AML and higher-risk types of MDS.

The study will be conducted in 2 parts (FIG. 2): Part 1 (Dose Escalation) and Part 2 (Cohort Expansion). Part 1 (Dose Escalation) is designed to determine RP2D(s) and dosing regimen(s) based on safety, PK, pharmacodynamics, and preliminary assessment of efficacy across several dose regimens; the RP2D(s) and dosing regimen(s) determined in Part 1 will be studied in additional participants in Part 2 to further assess their safety and efficacy in participants with specific subtypes of myeloid malignancies.

During the study, safety will be monitored at each dose escalation step and at regular intervals during cohort expansion. Decisions will be based on the review of all available data including, but not limited to PK, pharmacodynamic, safety, and preliminary antitumor activity, and as guided by the dose escalation rules.

The study will be initiated with the CD33×Vδ2 bispecific antibody by IV infusion at a dose based on the MABEL followed by dose escalation guided by BOIN design.

All participants must be hospitalized for observation for a minimum of 24 hours following IV infusion after the first dose and any subsequent dose increase through the first target dose of the CD33×Vδ2 bispecific antibody (starting after the EOI/EOF). If part of site practice, participants may receive study treatment in an outpatient infusion center followed by a direct transfer to an inpatient setting for the required observation period. Participants should be observed for at least 3 hours after each study drug administration through the first 4 target doses following administration (the observation period may be reviewed and adjusted as required).

The total number of participants enrolled is anticipated to be approximately 100, with approximately 60 DLT-evaluable participants in Part 1 and up to approximately 20 evaluable participants enrolled for each participant group/disease category cohort in Part 2.

### Part 1 (Dose Escalation)

Part 1 is designed to determine the RP2D(s) and dosing regimen(s) (for further study in Part 2) for the CD33×Vδ2 bispecific antibody, generally by testing successively higher doses, beginning at the MABEL-based starting dose of the antibody across cohorts of clinical trial participants while conducting real-time assessment of safety, efficacy, PK, and pharmacodynamics for each cohort. Dose escalation will be carried out in pre-approved cohorts.

The assessment of safety for a given cohort will include review and discussion of the adverse events for the participants in that cohort. Safety for a cohort will also be assessed by the proportion of participants in the cohort that experience a DLT. Laboratory data, PK, PD, biomarker data, and clinical activity for the participants in a given cohort may also be reviewed. Additional enrollment to further characterize the safety and preliminary efficacy of specific regimens may inform the regimen(s) to be explored in Part 2.

### BOIN Design for Part 1

A BOIN design will be implemented to support the dose escalation (Liu 2015; Yuan 2016)

### Cohort Enrollment

Dose escalation will be carried out in cohorts at pre-approved doses. The following guidelines apply during dose escalation:

Each cohort will have ≥1 DLT-evaluable participant. If a Grade ≥2 toxicity is observed, enrollment in that cohort and subsequent cohorts will require ≥3 DLT-evaluable participants.
Dose escalation cannot exceed half-log(10) (approximately three-fold) increments from the previous dose level.
If more than 1 participant is treated at a dose level, the first participant treated at that dose level must be observed for a minimum of 36 hours prior to treating subsequent participants.
CRS-mitigating strategies, including pretreatment medication, or postdose medications, may be incorporated following the first observation of a Grade ≥2 CRS event or any grade ICANS event.
Alternative dosing regimens may be tested based on emerging data.

### Provisional Dosing Table

A sample provisional dosing table is provided below (Table 6). Dose levels are subject to change based on emerging data. Intermediate dose level increments are possible to ensure the safety of study participants. A maximum dose level has not been identified for this study.

TABLE 6
Provisional Dosing Table

| Dose level | Increment |
|---|---|
| 1 | Starting dose (MABEL) |
| 2 | half-log(10) |
| 3 | half-log(10) |
| 4 | half-log(10) |
| 5 | half-log(10) |

### Determination of RP2D(s) and Dosing Regimen(s)

One or more RP2D(s) and dosing regimen(s) will be determined after review of available PK, pharmacodynamics, target engagement, efficacy, safety, and tolerability data across the cohorts. For the dose and schedule to be studied in Part 2 as a putative RP2D, there must be at least 6 participants studied in the DLT Evaluable Analysis Set in Part 1, and the BOIN dose escalation rule must not require de-escalation for that dose.

The RP2D(s) and dosing regimens may be studied further in additional participants in Part 2 (Cohort Expansion).

### Definition of Dose-Limiting Toxicity

Criteria for DLT are outlined in Table 7. Any modification of the DLT definition will require approval by Health Authorities.

TABLE 7
Dose-Limiting Toxicity Criteria

General
Any toxicity that requires discontinuation of treatment
Any Grade 5 toxicity
Non-hematologic Toxicity
Grade 3 or 4
Exceptions to Non-hematologic Toxicity Criteria^{a}
First occurrence Grade 3 CRS or IRR that improves to Grade ≤2 within 3 days
TLS Grade ≤4 that resolves in ≤5 days with optimal treatment and supportive care
Grade 3 fatigue, fever, constipation, nausea, vomiting, or diarrhea that improves to Grade ≤2 in <7
days with supportive care
Grade 3 hypertension controlled by oral medical management and improved to Grade ≤2 within 3
days
Grade ≥3 ALT/AST increases that resolve to Grade ≤2 or baseline in ≤7 days, unless Hy's Law criteria
are met^{b}
Grade 3 lipase or amylase increase without clinical or radiological evidence of pancreatitis
Grade ≥3 alkaline phosphatase, GGT, or electrolyte abnormalities that improve to Grade ≤2 or
baseline in ≤7 days or respond to supportive care
Hematologic Toxicity^{b}
Grade 4 neutropenia for >21 days despite supportive care according to institutional standards.
Febrile neutropenia without a documented infectious etiology Grade 4 for any duration or Grade 3
for >7 days despite supportive care.
Grade 4 anemia that is unexplained by underlying disease
Footnotes:
^{a}In case lab abnormal toxicity is a symptom of underlying syndrome (eg. CRS, TLS), the grading of syndrome supersedes lab abnormality. All observed exceptions will be reviewed to determine if any meet DLT criteria.
^{b}Given the disease-driven cytopenias in these patient populations, these hematologic toxicities will be considered DLTs in participants without simultaneous evidence of worsening disease.
Toxicity grading for CRS and ICANS is based on the ASTCT guidelines. Grading for all other AEs follows NCI-CTCAE.

### Part 2 (Cohort Expansion)

One or more RP2D(s) and dosing regimen(s) may be determined in Part 1 to be studied further in additional participants in Part 2 (Cohort Expansion).

In Part 2, cohorts of approximately 20 evaluable participants each may be enrolled for participants with R/R AML or R/R higher-risk types of MDS, respectively. Based on emerging data, specific disease-based subgroups may be added, dropped, or revised, as supported by emerging evidence of clinical activity. The objective of a cohort in Part 2 is to further characterize the safety as well as preliminarily evaluate the efficacy of a RP2D and dosing regimen in a particular disease category.

### Treatment Dosing Regimen

The study will be initiated with Q2W target doses based on the predicted human PK. Other dose regimens may be evaluated based on emerging safety, PK, and efficacy data.

### Study Population

Screening for eligible participants will be performed within 30 days before administration of the study treatment.

The inclusion and exclusion criteria for enrolling participants in this study are described below. If there is a question about these criteria, the investigator must consult with the appropriate sponsor representative and resolve any issues before enrolling a participant in the study. Waivers are not allowed. For a discussion of the statistical considerations of participant selection, refer to Sample Size Determination.

### Inclusion Criteria

Each potential participant must satisfy all of the following criteria to be enrolled in the study:

### Age

1. At the time of informed consent, be ≥18 years of age or at least the legal age of majority ii the jurisdiction in which the study is taking place.

### Disease Characteristics

2. Have a diagnosis, per the WHO 2022 criteria (WHO 2022), of
   a. AML or
   b. Moderate high, high, or very high risk MDS per the IPSS-M (Bernard 2022) (Appendix 2). If molecular results within the 12 months prior to informed consent are not available, participants must have IPSS-R >3.5, which corresponds with risk categories of intermediate, high-risk, or very high-risk MDS (Greenberg 2012) (Appendix2).

All participants must have relapsed or refractory disease after at least one line of approved therapy with no other approved therapies available that would be more appropriate in the investigator's judgement. Eligible participants will have a bone marrow blast burden ≥5%. Participants with newly transformed secondary AML may be enrolled at the investigator's discretion, provided that no standard therapeutic options exist based on the treatment prior to transformation.

### Weight

3. Body weight that is ≥40 kg.

### Performance Status

4. Have an ECOG performance status of 0-2 (Oken 1982).

### Hepatic and Renal Function

5. Have adequate renal function defined as CKD-EPIcr eGFR ≥40 mL/min computed with the calculator on the CKD-EPI website (http://ckdepi.org/equations/gfr-calculator/).
6. Participants are eligible if they have the following laboratory values:
   a. AST and ALT <3×ULN
   b. Serum total bilirubin <1.5×ULN (Bilirubin in case of known congenital nonhemolytic hyperbilirubinemias such as Gilbert's Syndrome may demonstrate isolated total bilirubin ≥1.5×ULN if conjugated [direct] bilirubin <1.5×ULN).

### Hematologic Values

7. Participants must have hematology laboratory parameters meeting the following criteria: WBC ≤20×10⁹/L. Hydroxyurea or a single dose of high-dose cytarabine may be used to lower WBC count during screening, and additional use of hydroxyurea is permitted during study treatment.

### Sex and Contraceptive/Barrier Requirements

8. While on study treatment and for 6 months after the last dose of study treatment, a participant must:
   Not breastfeed or be pregnant.
   Not donate gametes (ie, eggs or sperm) or freeze for future use for the purposes of assisted reproduction.
   If of childbearing potential,
      have a negative highly sensitive (eg, β-hCG) pregnancy test at screening and within 24 hours before the first dose of study treatment, and agree to further pregnancy tests,
      practice at least 1 highly effective method of contraception; if oral contraceptives are used, a barrier method of contraception must also be used.
   If a participant's partner is of childbearing potential,
      the partner must practice a highly effective method of contraception unless the participant is vasectomized.

### Informed Consent

9. Must sign an ICF (or their legally acceptable representative must sign) indicating that the participant understands the purpose of, and procedures required for, the study and is willing to participate in the study. Consent is to be obtained prior to the initiation of any study-related tests or procedures that are not part of standard of care for the participant's disease.
10. Be willing and able to adhere to the lifestyle restrictions specified in this protocol.

### Exclusion Criteria

Any potential participant who meets any of the following criteria will be excluded from participating in the study:

### Medical Conditions

1. Has a medical history of clinically significant pulmonary compromise, particularly the need for current supplemental oxygen use to maintain adequate oxygenation.
2. Has evidence of an uncontrolled systemic viral, bacterial, or fungal infection. Antimicrobial prophylaxis is permitted.
3. Has known allergies, hypersensitivity, or intolerance to the excipients of the antibody drug product
4. Had major surgery or had significant traumatic injury within 14 days of planned first dose of the CD33×Vδ2 bispecific antibody. Note: Participants with planned surgical procedures may participate.

### Prior Malignancies

5. Had a prior or concurrent second malignancy (other than the disease under study) with natural history or treatment likely to interfere with any study endpoints of safety or the efficacy of the study treatment(s).

### Cardiovascular Dysfunction

6. Had any of the following within 6 months prior to start of study treatment: severe or unstable angina, myocardial infarction, major thromboembolic events (eg, pulmonary embolism, cerebrovascular accident), clinically significant ventricular arrhythmias or heart failure New York Heart Association functional classification Class III-IV.
   Uncomplicated deep vein thrombosis is not considered exclusionary.

### Brain and Central Nervous System Metastases

7. Has known active central nervous system involvement.

### HIV Status

8. Is human immunodeficiency virus-positive and meets any of the following:
   a. Detectable viral load (ie, ≥50 copies/mL) at screening
   b. CD4+ count ≤300 cells/mm³ at screening
   c. AIDS-defining opportunistic infection within 6 months of screening
   d. Receives treatment other than continued HAART. A change in HAART due to resistance should occur at least 3 months prior to screening. A change in HAART due to toxicity is allowed up to 4 weeks prior to screening.
Note: HAART that could interfere with study treatment is excluded (consult the sponsor for a review of medications prior to enrollment).

### Viral Hepatitis Assessments

9. Has active hepatitis of infectious origin.
   a. Seropositive for hepatitis B: defined by a positive test for HBsAg. Participants with resolved infection (ie, participants who are HBsAg negative with positive antibodies to total anti-HBc must be screened using RT-PCR measurement of HBV DNA levels. Those who are RT-PCR positive will be excluded. Participants with serologic findings suggestive of HBV vaccination (anti-HBs positivity as the only serologic marker) AND a known history of prior HBV vaccination, do not need to be tested for HBV DNA by RT-PCR.
   b. Known hepatitis C infection or positive serologic testing for anti-HCV antibody.
   Participants with positive hepatitis C antibody due to prior resolved disease can be enrolled only if a confirmatory negative hepatitis C RNA test is obtained at screening or within 3 months prior to first dose of study treatment.
   Other clinically active liver disease of infectious origin.

### Prior/Concomitant Therapy or Clinical Study Experience

10. Received any of the following prior therapies:
   a. Treatment within 2 weeks or 5 half-lives, whichever is shorter, before the first administration of study drug with any of the following agents: chemotherapy, targeted therapy, immunotherapy (see below for checkpoint inhibitors), radiotherapy, or any investigational agents. Hydroxyurea or a single dose of high-dose cytarabine is permitted.
   b. Receipt within 3 months before the first administration of study drug: allogenic stem cell transplant, autologous stem cell transplant, non-CD33 directed CAR T-cell therapy, or any non-immune cell engaging CD33 targeted therapy. Participants who received a CD33 targeting CAR T-cell therapy or any CD33 targeting immune cell engaging therapy will be excluded but exceptions may apply for specific cohorts.
   c. Prior treatment with a checkpoint inhibitor such that the first dose of the CD33×Vδ2 bispecific antibody would occur within 5 half-lives.
11. Uses corticosteroids >10 mg daily prednisone equivalents:
   a. A short course (ie, >10 mg daily prednisone equivalents for <7 days) of corticosteroids is permitted. Inhaled or topical steroids, and adrenal replacement doses ≤10 mg daily prednisone equivalents are permitted.
   b. If corticosteroids were used to treat immune-related adverse events associated with prior therapy, ≥7 days must have elapsed since the last dose of corticosteroid.
12. Has toxicities from previous anticancer therapies not resolved to baseline levels or to Grade ≤1 or to Grade ≤2 for alopecia, peripheral neuropathy and vitiligo. Toxicities from previous anticancer therapies should have resolved to baseline levels, or to Grade 1 or less, or to Grade ≤2 for alopecia, peripheral neuropathy and vitiligo.
13. Received a prior solid-organ transplantation.
14. Requires ongoing therapy with a prohibited medication.
15. Received or plans to receive any live, attenuated vaccine within 4 weeks before the first dose of study drug or within 4 weeks after the last dose of study drug. Approved non-live and non-replication-competent vaccines or those authorized for emergency use (eg, COVID-19, Mpox) by local health authorities are allowed.

### Other Exclusions

16. Has active autoimmune disease that requires systemic immunosuppressive medications (e.g., chronic corticosteroid, methotrexate, or tacrolimus) or graft versus host disease that requires immunosuppressant therapy.
17. Has any condition for which, in the opinion of the investigator, participation would not be in the best interest of the participant (e.g., compromise the well-being) or that could prevent, limit, or confound the protocol-specified assessments.

NOTE: Investigators must ensure that all study enrollment criteria have been met at screening. If a participant's clinical status changes (including any available laboratory results or receipt of additional medical records) after screening but before the first dose of study treatment is given such that the participant no longer meets all eligibility criteria, then the participant must be excluded from participation in the study.

### Study Treatment and Concomitant Therapy

### Study Treatment(s) Administered

For this study, “study treatment” refers to the CD33×Vδ2 bispecific antibody and its diluent. The CD33×Vδ2 bispecific antibody is considered an IMP. All other study-specified medications are considered concomitant medications.

Study treatment administration must be captured in the source documents and the CRF.

The CD33×Vδ2 bispecific antibody drug product and diluent will be manufactured and provided under the responsibility of the sponsor. The study drug labels will contain information to meet the applicable regulatory requirements.

### Description of Treatment

TABLE 8
Administration of the CD33 × Vδ2 bispecific antibody

| | |
|---|---|
| Study Treatment | CD33 × Vδ2 bispecific antibody |
| Study treatment | Refer to the IPPI for preparation, handling, storage, and administration of the |
| instructions | CD33 × Vδ2 bispecific antibody. |
| Starting dose/dose | Dose escalation will be initiated at a starting dose IV. |
| level(s) | |
| Route of | IV infusion for each cohort. |
| administration | Refer to the correct IPPI and the SIPPM for complete details regarding study |
| | drug preparation. |
| Dosing Regimen | The study will be initiated with CD33 × Vδ2 bispecific antibody study drug |
| | administered every 2 weeks. |
| | After the participant's first scheduled disease assessment after start of |
| | treatment, the dosing regimen may be adjusted after discussion between the |
| | investigator and the sponsor team. |
| Dosage | Solution; CD33 × Vδ2 bispecific antibody in 50 mM histidine, 7.5% (w/v) |
| formulation | sorbitol, 100 mM L-methionine, and 0.06% (w/v) Polysorbate 20 at a pH of 6.0 |
| Premedication | No premedications are required, and the regimen may be adjusted based on |
| | emerging data. |
| Hospitalization | Participants must be hospitalized for a minimum of 24 hours following |
| | EOI/EOF following the first and any subsequent dose increase through the first |
| | target dose. |
| Observation Period | The observation period begins following IV infusion (after the EOI/EOF). All |
| | participants must be hospitalized for a minimum of 24 hours after the EOI/EOF |
| | after the first any subsequent dose increase through the first target dose of the |
| | CD33 × Vδ2 bispecific antibody. Participants should be observed for at least 3 |
| | hours after each study drug administration following target doses 2-4 (the |
| | observation period may be reviewed and increased or decreased as required). |

### Dose Modification

At scheduled treatment visits, the participant will be evaluated for possible toxicities. The primary methods for managing toxicities (any grade) are dose delay and dose reduction. Depending on the toxicity and the determination of causality, study drug may be reintroduced at the same or a lower dose as clinically indicated and in consultation with the Sponsor.

Any dose/dosage adjustment or planned change to the dosing regimen must first be discussed with the sponsor's medical monitor prior to implementing and must be overseen by medically qualified study site personnel (principal or subinvestigator unless an immediate safety risk appears to be present).

### Dose Modification and Re-Treatment Criteria for Non-Hematologic Toxicities

In case a dose modification is necessary following occurrence of non-hematologic toxicities, the study treatment will be administered as noted in Table 9. If a toxicity requires dose reduction of study drug, the dose should be reduced at least to the next lower dose level that has been determined safe.

TABLE 9
Dose Modification Guidance for Non-hematologic Toxicity^{a}

| Grade | Occurrence | Treatment Modification |
|---|---|---|
| Grade 2 | Any | If toxicity is intolerable, study treatment should be withheld until |
| | | adverse event returns to Grade ≤1 or baseline. |
| Grade 3 | 1^{st} | Hold study treatment until resolution to Grade ≤1 or baseline. |
| | occurrence | Treatment may be resumed at the same dose and schedule. |
| | 2^{nd} | Hold study treatment until resolution to Grade ≤1 or baseline. |
| | occurrence | Consider dose reduction by 1 dose level. |
| | 3^{rd} | Discontinue study treatment. Further treatment may be considered |
| | occurrence | but only after consultation with and approval by the Sponsor. |
| | Exceptions: | |
| | First occurrence CRS, ICANS, cerebral edema/increased intracranial pressure, | |
| | IRR | |
| | TLS Grade ≤4 that recovers in ≤5 days | |
| | Fatigue, fever, constipation, nausea, vomiting, or diarrhea lasting <7 days with | |
| | best supportive care | |
| | Systolic blood pressure >160 of diastolic blood pressure >100 mm Hg that can | |
| | be controlled by medical management | |
| | ALT or AST that resolves to Grade ≤2 or baseline within 7 days, unless | |
| | criteria for Hy's law are met | |
| | Lipase or amylase increase not associated with evidence of pancreatitis | |
| | Alkaline phosphatase, GGT, or electrolyte abnormalities that resolve to Grade ≤1 | |
| | or baseline within 7 days or respond to conventional medical interventions | |
| | Electrolyte abnormalities that persist ≤72 hours and resolve Grade ≤1 or | |
| | baseline or respond to conventional medical interventions | |
| | Other clinically insignificant laboratory abnormalities or adverse events that | |
| | can be managed adequately with supportive care | |
| Grade 4 | Any | Hold study treatment until resolution to Grade ≤1 or baseline. |
| | | Treatment cannot be restarted unless agreed upon after consultation |
| | | with and approval by the sponsor. Retreatment will require a dose |
| | | reduction to the next approved lower target dose level. |

Footnotes:
^{a}If study drug is held due to laboratory abnormality, laboratory parameters must be rechecked as clinically indicated (and at minimum once a week) until they meet retreatment criteria listed in this table. In addition, if a toxicity is part of a syndrome, the grade of the syndrome determines the need for dose modification.

In addition, treatment must be permanently discontinued in the event of the following toxicities:
A second occurrence of Grade 3 ICANS.
A second consecutive occurrence of Grade 3 CRS during target dosing
Grade 4 CRS, ICANS, or any other non-hematologic Grade 4 event considered a life-threatening event related to study treatment.

### Retreatment Criteria for Hematologic Abnormalities

Hold criteria for bleeding, anemia, and neutropenia are outlined in Table 10. Transfusions for disease-related cytopenias are allowed throughout the treatment phase. Note that no therapy interruptions are indicated for isolated neutropenia (any grade) unless there is a neutropenia toxicity that qualifies as a DLT.

Supportive care for cytopenias in participants with AML or MDS, including transfusions or growth factors, should be provided in accordance with published clinical practice guidelines and standard institutional practice (NCCN AML 2023; NCCN MDS 2023). Therapy interruption guidelines for various hematologic abnormalities, either due to disease or treatment toxicities, are outlined in Table 10 and for neutropenia.

TABLE 10
Dose Modification for Hematologic Abnormalities

| Event | Treatment Modification |
|---|---|
| Anemia | Treatment with the CD33 × Vδ2 bispecific antibody should be held for Grade 4 |
| | anemia and can be resumed when the anemia improves to Grade ≤3. |
| Bleeding | Treatment with the CD33 × Vδ2 bispecific antibody should be held for Grade ≥2 |
| | bleeding and may be resumed once the bleeding improves to Grade ≤1. |
| Febrile | The CD33 × Vδ2 bispecific antibody should be held in the setting of febrile |
| Neutropenia | neutropenia, and treatment resumption may occur after fever resolution at the |
| | discretion of the treating physician. If a causative microbe is identified, the AE |
| | should be reclassified under the applicable infection term. Note: Grade 4 febrile |
| | neutropenia considered a life-threatening event related to study treatment will result |
| | in permanent study treatment discontinuation. |

If there is a treatment delay >28 days, retreatment with the CD33 × Vδ2 bispecific antibody will require medical monitor approval. Transfusions for disease-related cytopenias are allowed throughout the treatment phase.

### Dose Delay

If a dose is delayed, the subsequent doses are to be delayed ensuring a minimum 10-day interval between Q2W target doses. If the treatment regimen is revised from the initial Q2W dosing regimen, the sponsor will provide further guidance.

Treatment may be held for a maximum of 28 days from the date of the originally planned dose. Retreatment after a holding period of more than 28 days may only occur after approval by the sponsor's medical monitor or delegate who will also provide further dosing instructions.

### Dose Reductions

Guidelines for dose modifications and re-treatment criteria following occurrence of general toxicities and hematologic abnormalities listed in Table 9 and Table 10. If a toxicity requires dose reduction the dose should be reduced to the previous lower dose level deemed to be safe, or by a further decrease as recommended by the Sponsor's medical monitor or delegate. If the dose level associated with the toxicity and the next lower dose level are both deemed safe, the dose reduction for the participant may be an intermediate dose between the two dose levels upon sponsor approval. Dosing frequency reductions may also be approved by the Sponsor.
After the dose of study drug has been reduced it cannot be increased to a higher dose level without approval from the sponsor's medical monitor.
Treatment will be discontinued if toxicity meets the pre-established criteria.

### Intra-participant Dose Escalation

Participants enrolled in Part 1 Dose Escalation may be moved to a higher dose level deemed safe when the following conditions are met:
The participant has completed the DLT Evaluation Period.
The participant has tolerated their current dose level (eg, typically with Grade ≤2 toxicity).
The investigator and the Sponsor agree that dose escalation is supported by available data.
The participant agrees to dose escalation.

### Statistical Analyses

### Primary Endpoint

The primary endpoint is the incidence and severity of AEs, including DLT(s) in Part 1.

### Secondary Endpoints

Serum concentration-time profiles and PK parameters for the CD33×Vδ2 bispecific antibody including but not limited to AUCτ, Cₘₐₓ, and C_{trough}.
Presence of ADAs of the CD33×Vδ2 bispecific antibody
Overall response (MDS), complete response (AML and MDS), time to response, duration of response, and transfusion independence. Response will be assessed according to:
   ELN recommendations for AML (Döhner 2022; see Appendix 2)
   Revised IWG response criteria for higher-risk types of MDS (Zeidan 2023, Platzbecker 2019)

### Analysis Methods

ORR and CR will be tabulated, together with its 2-sided 95% exact confidence interval. In addition, the number and percentage of participants in each response category will be tabulated.

Time to response and duration of response will be analyzed using Kaplan-Meier estimates analysis. A summary table including number of participants included in the analysis, 25th and 75th percentiles, and median time-to-event (all with 95% CIs) will be presented. Kaplan-Meier estimates of the survival probability at appropriate time points may be provided. The data will be presented graphically using the Kaplan-Meier estimate of the survival function.

### Endpoint Definitions

The efficacy endpoints are:
Overall Response: OR is achieved when a participant with MDS has a CR (any type), PR, or HI according to the criteria in Appendices 0 and 0. Response to treatment with the study drug will be evaluated by the investigator. Overall Response will be summarized by ORR.
Complete Response: CR is achieved when a participant has a best response of CR (any type) according to the criteria in in Appendices 0 and 0. Complete Response for AML and MDS will be summarized by the CR rate.
OR Duration of Response (MDS only): OR DOR is defined for participants who achieved CR (any type), PR, or HI as the time between the date of initial documentation of the qualifying response to the date of first documented evidence of relapse, disease progression, initiation of a new systemic anti-cancer therapy (besides HSCT), or death, whichever comes first.
CR Duration of Response (AML and MDS): CR DOR is defined for participants who achieved CR (any type) as the time between the date of initial documentation of the CR (or subtype) to the date of first documented evidence of non-CR response (eg, MLFS), relapse, initiation of a new systemic anti-cancer therapy (besides HSCT), or death, whichever comes first.
OR Time to Response (MDS only): OR TTR is defined for participants who achieved CR (any type), PR, or HI as the time from the first dose of study drug to first qualifying response.
CR Time to Response (AML and MDS): CR TTR is defined for participants who achieved CR (any type) as the time from the first dose of study drug to first qualifying response.
Transfusion independence is defined as the absence of RBC and platelet transfusions for 8 weeks or longer after starting study treatment for participants with AML and 16 weeks or longer for participants with MDS. Participants with blood counts that meet the minimum parameters for use of transfusions but do not receive transfusions per investigator decision will not be counted as achieving TI.

Transfusions for hemoglobin ≥9.0 g/dL or for platelets ≥50×10⁹/L will not be counted as part of the TI assessment.

RBC transfusion dependence at baseline definitions:
AML: One or more RBC transfusions given in response to hemoglobin levels of ≤9.0 g/dL within 28 days prior to the start of study treatment (FDA Guidance for Industry, Acute Myeloid Leukemia: Developing Drugs and Biological Products for Treatment, 2020).
Higher-risk types of MDS: Three or more RBC transfusions given in response to hemoglobin levels of ≤9.0 g/dL in the 16-week period prior to the start of study treatment (Platzbecker 2019). If a 16-week period is not feasible, TD will be considered for participants with ≥2 RBC transfusions in response to hemoglobin levels of ≤9.0 g/dL an 8-week period prior to study treatment initiation.

Platelet transfusion dependence for all participants is defined as receipt of any platelet transfusion within 28 days prior to the start of study treatment (FDA Guidance for Industry, Acute Myeloid Leukemia: Developing Drugs and Biological Products for Treatment, 2020).

### Exploratory Endpoints

Determination of biomarkers of response to the CD33×Vδ2 bispecific antibody which may include mutation and expression profiles, T-cell frequency, activation, expansion, and characterization, cytokine/chemokine profile, and immune cell subsets associated with response/resistance.
Evaluation of relationships between PK, pharmacodynamics, biomarkers, AE profiles, and clinical activity or resistance to the CD33×Vδ2 bispecific antibody
Evaluation of RO of Vδ2 and CD33
During Part 2, MRD assessments may be completed using an immunophenotyping and/or molecular based approach. For flow cytometry MRD assay the cutoff for MRD negativity will be less than 0.1% and for molecular MRD assay the limited of detection will be ≤10⁻³, as recommended by the ELN working group (Döhner 2022, Appendix 2). In addition, other cutoff values will be evaluated.

### Safety Analyses

All safety analyses will be made on the Safety Analysis Set. The baseline value for safety assessment is defined as the value collected at the time closest to, but prior to, the start of the first study drug administration. The safety parameters to be evaluated are the incidence, severity, and type of AEs, clinically significant changes in the participant's physical examination findings, vital signs measurements, clinical laboratory, and other clinical test results (eg, ECG). Exposure to the study drug and reasons for discontinuation of study drug will be tabulated. AEs will be summarized by system organ class, preferred term, worst grade experienced, and dose level.

### Immunogenicity Analyses

The incidence of anti-CD33×Vδ2 antibodies will be summarized for all participants who receive ≥1 dose of the CD33×Vδ2 bispecific antibody and have appropriate samples for detection of antibodies to the CD33×Vδ2 bispecific antibody (ie, participants with at least 1 sample obtained after their first dose of the CD33×Vδ2 bispecific antibody).

A listing of participants who are positive for antibodies to the CD33×Vδ2 bispecific antibody will be provided. The maximum titers of antibodies to the CD33×Vδ2 bispecific antibody will be summarized for participants who are positive for antibodies to the CD33×Vδ2 bispecific antibody. Other immunogenicity analyses may be performed to further characterize the immune responses that are generated.

### Supporting Documentation and Operational Considerations

### Appendix 1: Response Assessment in AML

Response Assessment in AML

| | |
|---|---|
| Category | Definition |

Response options for disease assessments: Responses are ordered from best to worst through “non-
evaluable for response,” and the best applicable response should be selected for the participant.

| Complete remission (CR) ^{a} | Bone marrow blasts <5%; | |
|---|---|---|
| | Absence of circulating blasts; | |
| | Absence of extramedullary disease; | |
| | Absolute neutrophil count (ANC) ≥1.0 × 10⁹/L (1,000/μL); | |
| | Platelet count ≥100 × 10⁹/L (100,000/μL) | |
| CR with partial hematologic | BM, circulating blast, and EMD criteria of CR are met with BOTH: | |
| recovery (CRh) ^{a} | ANC ≥0.5 × 10⁹/L (500/μL) AND | |
| | platelet count ≥50 × 10⁹/L (50,000/μL) | |
| CR with incomplete | BM, circulating blast, and EMD CR criteria are met but with either | |
| hematologic recovery (CRᵢ) | residual neutropenia (<1.0 × 10⁹/L [1,000/μL]) OR thrombocytopenia | |
| | (<100 × 10⁹/L [100,000/μL]) | |
| Morphologic leukemia-free | Both BM and circulating blast and | Note: Marrow should not merely be |
| state (MLFS) | EMD CR criteria are met but No | “aplastic”; bone marrow spicules |
| | hematologic recovery required | should be present; ≥200 cells should |
| | | be enumerated in the aspirate or |
| | | cellularity should be at least 10% in |
| | | the biopsy. |

| | |
|---|---|
| Partial remission (PR) | All hematologic criteria of CR; |
| | Decrease of bone marrow blast percentage to 5% to 25%; |
| | AND decrease of pretreatment bone marrow blast percentage by at |
| | least 50% |
| No response ^{b} | Evaluable for response but not meeting the criteria for CR, CRh, CRi, |
| | MLFS, or PR. |
| Non-evaluable for response | This category includes patients without an adequate response evaluation |
| | (eg, death or withdrawal prior to response assessment, technically |
| | suboptimal bone marrow sample precluding assessment). |
| CR, CRh, or CRi without | CR, CRh or CRi with MRD below a defined threshold for a genetic |
| MRD (CR_{MRD-}, CRh_{MRD-} or | marker by qPCR, or by multiparametric flow cytometry. Response without |
| CRi_{MRD-}) ^{c} | MRD should be confirmed with a subsequent assessment at least 4 wks |
| | apart. The date of response without MRD is the first date in which the |
| | MRD was below the defined threshold |

Treatment failure

| | |
|---|---|
| Refractory disease | No CR, CRh or CRi at the response landmark.^{d} |
| Relapsed disease (after CR, | Bone marrow blasts ≥5%; or reappearance of blasts in the blood in ≥2 |
| CRh or CRi) | peripheral blood samples at least one week apart; or development of |
| | extramedullary disease |
| MRD relapse (after CR, | Conversion from MRD negativity to MRD positivity (method |
| CRh or CRi without MRD) ^{e} | independent) or Increase of MRD copy numbers ≥1 log₁₀ between any |
| | two positive samples in patients with CR_{MRD-LL}, CRh_{MRD-LL} or CRi_{MRD-LL} |
| | by qPCR. |

^{a} For patients with CR, CRh, or CRi, the presence of a low percentage of circulating blasts in the blood may represent a regenerating marrow and should not be interpreted as persistent disease. In such cases the blasts generally disappear within a week.
^{b} In addition, participants with a prior MLFS or PR who lose this response will be designated as having no response at the corresponding disease assessment.
^{c} MRD testing may be completed locally and is reported separately from the response assessment in the eCRF.
Response with MRD detection at low-level (CR_{MRD-}LL) is included in this category of CR, CRh or CRi without MRD. CR_{MRD-}LL is currently only defined for NPM1-mutant and CBF-AML
^{d}Multiparameter flow cytometry-MRD positivity is defined as ≥0.1% of CD45 expressing cells with the target immunophenotype. MRD test positivity by qPCR is defined as cycling threshold (Ct) <40 and is negative if Ct ≥40 in ≥2 of 3 replicates. In NPM1-mutated and CBF-AML, CR with molecular MRD detectable at low-level (CR_{MRD-}LL) defined as <2% is designated as negative for MRD, because when measured at the end of consolidation treatment, is associated with a very low relapse rate.
Source: Döhner 2022

### Appendix 2: Response Assessment in MDS

Response Assessment in MDS: Responses are ordered from best to worst, with the exclusion of the hematologic improvement categories, and the best applicable response should be selected for the participant.

| Category | Response Criteria (responses must last ≥4 weeks) | |
|---|---|---|
| Complete remission | Bone marrow: <5% myeloblasts; dysplasia may persist | |
| (CR) | Peripheral blood: Hb ≥10 g/dL; platelets ≥100 × 10⁹/L; | |
| | neutrophils | |
| | ≥1.0 × 10⁹/L; blasts 0% ^{a} | |
| Complete remission | Bone marrow: <5% myeloblasts; dysplasia may persist | |
| with limited count | Peripheral blood: blasts 0% ^{a} and | |
| recovery (CR_{L}) | CR_{bi}: not meeting CR but only 2 of the following: Hb ≥10 | |
| CR_{bi} (bilineage) | g/dL; platelets ≥100 × 10⁹/L; neutrophils ≥1.0 × 10⁹/L | |
| CRᵤₙᵢ (unilineage) | CRᵤₙᵢ: not meeting CR but only 1 of the following: Hb ≥10 | |
| | g/dL; platelets ≥100 × 10⁹/L; neutrophils ≥1.0 × 10⁹/L | |
| Complete remission | Bone marrow: <5% myeloblasts; dysplasia may persist | |
| with partial hematologic | Peripheral blood: Not meeting criteria for CR or CR_{L}; no Hb | |
| recovery (CRh) | threshold required; platelets ≥50 × 10⁹/L; neutrophils ≥0.5 × 10⁹/L; | |
| | blasts 0% ^{a} | |
| Partial remission | All CR criteria except bone marrow blasts decreased by ≥50% | |
| (PR) | over pretreatment but still >5%; Cellularity and morphology not | |
| | relevant | |
| HI-E; HI-P; HI-N | Not meeting criteria for CR or CR_{L} | |
| Hematologic | Baseline | HI-E confirmed based on baseline status |
| improvement^{b,c}- | transfusion burden | NTD: ≥2 consecutive hemoglobin ≥1.5 g/dL ≥8 |
| erythroid (HI-E) | categories | wks in a 16-24 week period compared with the |
| | NTD: 0 RBCs in | lowest mean of 2 hemoglobin measurements |
| | 16 wks ^{d} | (omitting transfusions) within 16 wks prior to |
| | LTB: 3-7 RBCs | study therapy. |
| | in 16 wks in ≥2 | LTB: 0 RBCs in ≥8 wks within a 16-24 week |
| | transfusion | period |
| | episodes, ≤3 in 8 | HTB |
| | wks ^{d} | Major response: 0 transfusions in ≥8 wks |
| | HTB: ≥8 RBCs | within a 16-24 week period |
| | in 16 wk, ≥4 in 8 | Minor response: reduction by ≥50% of RBCs |
| | wks | within ≥16 wks |
| | | Note: Response durations ≥16 wks are |
| | | considered clinically meaningful. |

| | |
|---|---|
| Hematologic | For patients with pretreatment platelet count ≤100 × 10⁹/L |
| improvement^{b,e}- | Increase of ≥30 × 10⁹/L (starting with >20 × 10⁹/L) or |
| platelet (HI-P) | Increase from <20 × 10⁹/L to >20 × 10⁹/L AND by ≥100% |
| | In addition, evolution of bleeding symptoms is to be taken into |
| | account and increments of platelets into the normal range for patients |
| | with pretreatment platelets of ≥100 × 10⁹/L are to be reported. |
| Hematologic | For patients with pretreatment neutrophil count <1 × 10⁹/L: |
| improvement^{b}- | Increase of ≥100% and absolute increase of >0.5 × 10⁹/L |
| neutrophil (HI-N) | Increments of neutrophils for patients with a pretreatment ANC |
| | of ≥1.0 × 10⁹/L are to be reported |
| No response | Not meeting criteria for CR, CR_{L}, CRh, PR, or HI |
| Not evaluable | This category may include patients yet to have a response |
| | assessment, suffering early death, exiting the study early, or those with |
| | a technically suboptimal BM sample precluding assessment. |
| Progressive disease | Fulfilling any of the criteria below: |
| (PD) ^{g} | Disease progression by blasts: ≥50% relative increase in blasts and |
| | absolute increase of blast percentage by at least 5% from |
| | pretreatment sample taken before current line of therapy. |
| | Disease progression by worsening cytopenia: new, repeated (more |
| | than once and separated by ≥7 days) need for RBC or platelet |
| | transfusions within 8 weeks, not related to acute intercurrent illness |
| | or treatment effect, in the absence of HI of at least one other blood |
| | lineage as defined above. |
| | Progression to AML ^{f}: ≥50% increase in blasts from baseline |
| | assessment to ≥20% blasts. |
| Disease | Fulfilling any of the criteria below: |
| relapse (after CR, CR_{L}, | Disease relapse by blasts: absolute and relative increase in BM |
| CRh, PR, or HI) ^{g} | blasts by at least 5% and ≥50%, respectively, from prior |
| | assessment, or reappearance of blasts in the blood, or development |
| | of extramedullary disease (myeloid sarcoma). |
| | Disease relapse by worsening cytopenias: decrement in one or more |
| | blood cell lineage counts by ≥50% from maximum |
| | remission/response levels for platelets or absolute neutrophil count |
| | or a reduction of Hb by 1.5 g/dL combined with an absolute |
| | reduction in the same lineage(s) as follows: Hb <10 g/dL, |
| | platelets <100 × 10⁹/L, or absolute neutrophils <1.0 × 10⁹/L or repeated |
| | (more than once and separated by ≥7 days) need for RBC or |
| | platelet transfusions which are not related to acute intercurrent |
| | illness or treatment effect; in the absence of HI of at least one other |
| | blood lineage as defined above. |

^{a} If there is a discrepancy between <5% blasts in the bone marrow with peripheral blasts >0% at time of the bone marrow evaluation, repeat the peripheral blast assessment within 2 weeks. If the peripheral blasts clear within this window, the patient will have achieved a CR.
^{b}Screening period for the evaluation of transfusion burden and baseline Hb levels is ideally 16 weeks, but an 8-week screening period before treatment initiation is acceptable. For HI and TI assessments, a 16-week time window should be used. Hematologic improvements will be defined as cell lineage(s) achieving recovery at each disease assessment.
^{c}If treatment interruptions/reductions following HI-E result in apparent loss of HI-E, if the response resumes with therapy re-introduction (even at a lower dose), then the transient platelet drop will not count as a loss of HI-E.
^{d} IWG does not define the categorization of recipients of 1 or 2 RBC during the 16-week pre-treatment period, so this scenario will be considered within LTB for this study. To achieve HI-E, in addition to meeting the LTB HI-E criteria, these participants must also demonstrate a hemoglobin increase ≥1.5 g/dL.
^{e}Dose-adjustment policy for platelet counts on treatment:
If treatment interruptions/reductions following HI-P result in apparent loss of HI-P, if the response resumes with therapy re-introduction (even at a lower dose), then the transient platelet drop will not count as a loss of HI-P.
Treatment interruptions/reductions must be accompanied by at least weekly platelet monitoring.
Two subsequent platelet counts >450 × 10⁹/L are sufficient to discontinue any thrombopoietin agonist therapy.
^{f} For patients with an absolute BM blast increase to ≥20% but who have <50% relative BM blast count increase from pretreatment before current line of therapy, this could denote progression in the right clinical context where additional therapeutic options may be available with a new diagnosis of AML.
^{g} Patients with either PD or disease relapse (after CR, CR_{L}, CRh, PR, or HI) are considered to have Treatment Failure at the time of either of these confirmed responses.
Source: Zeidan 2023; Platzbecker 2019

### REFERENCES

Amgen. Amgen reports fourth quarter and full year 2020 financial results. Feb. 2, 2021. [press release]. Available at: https://www.amgen.com/newsroom/press-releases/2021/02/amgen-reports-fourth-quarter-and-full-year-2020-financial-results. Accessed Apr. 12, 2024.
Amgen. Amgen reports first quarter 2022 financial results. Apr. 27, 2022. [press release]. Available at: https://www.amgen.com/newsroom/press-releases/2022/04/amgen-reports-first-quarter-2022-financial-results. Accessed Apr. 12, 2024.
Bernard E, Tuechler H, Greenberg P, et al. Molecular International Prognostic Scoring System for Myelodysplastic Syndromes. NEJM Evidence 2022; 1(7); doi:10.1056/EVIDoa2200008.
Cheson B D, Greenberg P L, Bennett J M, et al. Clinical application and proposal for modification of the International Working Group (IWG) response criteria in myelodysplasia. Blood. 2006; 108(2):419-425.
Döhner H, Wei A H, Appelbaum F R, et al. Diagnosis and management of AML in adults: 2022 recommendations from an international expert panel on behalf of the ELN. Blood 2022; 140 (12):1345-1377.
European Medicines Agency. Guideline on strategies to identify and mitigate risks for first-in-human and early clinical trials with investigational medicinal products (EMEA/CHMP/SWP/28367/07 Rev. 1), July 2017
Greenberg P L, Tuechler H, Schanz J, et al. Revised international prognostic scoring system for myelodysplastic syndromes. Blood. 2012 Sep. 20; 120(12):2454-2465.
Kater A P, Van De Donk N W C J, Rodriguez-Otero P, et al. Lava-051, a novel bispecific gamma-delta T-cell engager (Gammabody), in relapsed/refractory MM and CLL: pharmacodynamic and early clinical data. Blood. 2022; 140(Supplement 1):4608-4609.
Lameris R, Ruben J M, de Weerdt I, et al. Bispecific Vγ9Vδ2-T and type 1 NKT cell engager Lava-051 as first-in-class clinical candidate to target CD1d expressing CLL, MM and AML. Blood. 2021; 138(Supplement 1):2266.
Lameris R, Ruben J, Roovers R, et al. 686: Mechanism of action of LAVA-051, a bispecific Vγ9Vδ2 T-cell engager (bsTCE), confirmed in the clinical setting. J Immunother Cancer. 2022:10(Suppl 2):A716.
Liu S, Yuan Y. Bayesian optimal interval designs for phase I clinical trials. J R Stat Soc Ser C Appl Stat. 2015; 64(3):507-523.
Narayan R, Piérola A A, Donnellan W, et al. First-in-human Study of JNJ-67571244, a CD33/CD3 Bispecific Antibody, in Relapsed/Refractory Acute Myeloid Leukemia and Myelodysplastic Syndrome. Clin Trans Sci. 2024; 17:e13742.
[NCCN] National Comprehensive Cancer Network. Clinical practice guidelines in oncology: Acute myeloid leukemia. Version 6.2023. Available at: https://www.nccn.org/professionals/physician_gls/pdf/aml.pdf. Accessed Jan. 12, 2024.
[NCCN] National Comprehensive Cancer Network. Clinical practice guidelines in oncology: Myelodysplastic syndromes. Version 2.2023. Available at: https://www.nccn.org/professionals/physician_gls/pdf/mds.pdf. Accessed Jan. 12, 2024.
Nguyen K A, Hamzeh-Cognasse H, Palle S, et al. Role of Siglec-7 in apoptosis in human platelets. PLoS One. 2014 Sep. 17; 9(9):e106239.
Nicoll G, Ni J, Liu D, et al. Identification and characterization of a novel siglec, siglec-7, expressed by human natural killer cells and monocytes. J Biol Chem. 1999 Nov. 26; 274(48):34089-95.
Oken M M, Creech R H, Tormey D C, et al. Toxicity and response criteria of the Eastern Cooperative Oncology Group. Am J Clin Oncol. 1982; 5:649-655.
Park J H, Lee H K. Function of γδ T cells in tumor immunology and their application to cancer therapy. Exp Mol Med. 2021; 53(3):318-327
Platzbecker U, Fenaux P, Ades L, et al. Proposals for revised IWG 2018 hematological response criteria in patients with MDS included in clinical trials. Blood 2019; 133:1020-1030.
Prebet T, Zeidan A. Trends in Clinical investigation for myelodysplastic syndromes. Clinical Lymphoma Myeloma and Leukemia. 2016; 16:S57-S63.
Schanz J, Tuchler H, Sole F, et al. New comprehensive cytogenetic scoring system for primary myelodysplastic syndromes (MDS) and oligoblastic acute myeloid leukemia after MDS derived from an international database merge. J Clin Oncology 2012; 30:820.
Van De Vyver A J, Marrer-Berger E, Wang K, Lehr T, Walz A C. Cytokine Release Syndrome By T-cell-Redirecting Therapies: Can We Predict and Modulate Patient Risk?Clin Cancer Res. 2021 Nov. 15; 27(22):6083-6094.
[WHO] World Health Organization. Classification of Tumours Editorial Board. Haematolymphoid tumours. Lyon (France): International Agency for Research on Cancer; 2022 (WHO classification of tumours series, 5th ed.; vol. 11). Available from: https://tumourclassification.iarc.who.int/chapters/63. Accessed Jan. 4, 2024.
Yuan Y, Hess K R, Hilsenbeck S G, Gilbert M R. Bayesian Optimal Interval Design: A Simple and Well-Performing Design for Phase I Oncology Trials. Clin Cancer Res. 2016; 22(17):4291-4301.
Zeidan A, Platzbecker U, Bewersdorf J P, et al. Consensus proposal for revised International Working Group 2023 response criteria for higher-risk myelodysplastic syndromes. Blood 2023; 141:2047-2061

1. A unit dose of a CD33×Vδ2 bispecific antibody, wherein said unit dose is selected from the group consisting of: from 7 milligrams (mg) to 70 mg, 70 mg, 69 mg, 68 mg, 67 mg, 66 mg, 65 mg, 64 mg, 63 mg, 62 mg, 61 mg, 60 mg, 59 mg, 58 mg, 57 mg, 56 mg, 55 mg, 54 mg, 53 mg, 52 mg, 51 mg, 50 mg, 49 mg, 48 mg, 47 mg, 46 mg, 45 mg, 44 mg, 43 mg, 42 mg, 41 mg, 40 mg, 39 mg, 38 mg, 37 mg, 36 mg, 35 mg, 34 mg, 33 mg, 32 mg, 31 mg, 30 mg, 29 mg, 28 mg, 27 mg, 26 mg, 25 mg, 24 mg, 23 mg, 22 mg, 21 mg, 20 mg, 19 mg, 18 mg, 17 mg, and 16 mg, 15 mg, 14 mg, 13 mg, 12 mg, 11 mg, 10 mg, 9 mg, 8 mg, 7 mg; and
wherein the CD33×Vδ2 bispecific antibody comprises the polypeptides set forth in SEQ ID NOs: 33 and 34.

2. The unit dose of the CD33×Vδ2 bispecific antibody of claim 1, wherein the CD33×Vδ2 bispecific antibody comprises, instead, the polypeptides set forth in SEQ ID NOs: 35 and 36.

3. The unit dose of the CD33×Vδ2 bispecific antibody of claim 1, wherein the unit dose of the CD33×Vδ2 bispecific antibody is comprised in a pharmaceutical composition at an antibody concentration of 2 mg/mL, 20 mg/mL, 30 mg/mL, or 70 mg/mL.

4. The unit dose of the CD33×Vδ2 bispecific antibody of claim 3, wherein the pharmaceutical composition at an antibody concentration of 2 mg/mL, is further diluted with a vehicle to a unit dose between 0.017 mg and 8 mg, wherein the vehicle optionally comprises 0.2 mg/mL recombinant human albumin (rHA) in 10 mM acetate, 0.04% (w/v) polysorbate 20, 50 mM NaCl, 5.7% (w/v) sucrose, at a pH of 5.2.

5. The unit dose of the CD33×Vδ2 bispecific antibody of claim 3, wherein the pharmaceutical composition at an antibody concentration of 2 mg/mL, is further diluted with normal saline to a unit dose between 8 mg and 60 mg.

6. The unit dose of the CD33×Vδ2 bispecific antibody of claim 3, wherein the pharmaceutical composition at an antibody concentration of 20 mg/mL or 30 mg/mL, each independently, is further diluted with normal saline to a unit dose between 7 mg and 70 mg.

7. The unit dose of the CD33×Vδ2 bispecific antibody of claim 3, wherein the pharmaceutical composition at an antibody concentration of 70 mg/mL, is further diluted with normal saline to a unit dose between 60 mg and 385 mg.

8. The unit dose of the CD33×Vδ2 bispecific antibody of claim 1, wherein the unit dose is adapted for delivery once every two weeks (Q2W), or once every week (Q1W).

9. The unit dose of the CD33×Vδ2 bispecific antibody of claim 1, wherein the unit dose is adapted for intravenous delivery.

10. The unit dose of the CD33×Vδ2 bispecific antibody of claim 1, wherein the unit dose is adapted for delivery by infusion during 0.5-4 hours, or about 1 hour.

11. A pharmaceutical composition comprising: (i) the unit dose of the CD33×Vδ2 bispecific antibody according to claim 1, and (ii) a pharmaceutically acceptable carrier.

12. The pharmaceutical composition according to claim 11, wherein the CD33×Vδ2 bispecific antibody is present at a concentration of 2 mg/mL, 20 mg/mL, 30 mg/mL, or 70 mg/mL.

13. The pharmaceutical composition according to claim 11, wherein the pharmaceutically acceptable carrier comprises:
a buffer present at a concentration within the range from 10 mM to 80 mM, wherein the buffer comprises histidine, acetate, citrate, or phosphate;
a tonicifying agent present at a concentration within the range from 2 to 10% (w/v), wherein the tonicifying agent is one or more sugar and/or polyol comprising sorbitol, sucrose, trehalose, glycerol, mannitol, or dextrose;
a surfactant present at a concentration within the range from 0.04 to 0.2% (w/v), wherein the surfactant comprises polysorbate 20, polysorbate 80, or poloxamer 188;
a stabilizer present at a concentration within the range from 20 to 120 mM, wherein the stabilizer is L-methionine;
wherein the formulation has a pH within the range from 5 to 8 when in aqueous form.

14. The pharmaceutical composition according to claim 11, wherein the pharmaceutically acceptable carrier comprises:
50 mM histidine;
7.5% (w/v) sorbitol;
0.06% (w/v) polysorbate 20;
100 mM of L-methionine;
wherein the formulation has a pH of 6 when in aqueous form.

15. The pharmaceutical composition of claim 11, where said pharmaceutical composition is adapted for intravenous administration.

16. The pharmaceutical composition of claim 11, where said pharmaceutical composition is in the form of a vial, syringe, syringe pump, infusion pump, or an intravenous bag.

17.-22. (canceled)

23. A method of treating Acute Myeloid Leukemia (AML) or Myelodysplastic Neoplasms (MDS) in a patient, said method comprising administering to the patient in need thereof the unit dose of the CD33×Vδ2 bispecific antibody of claim 1.

24. The method of treating AML or MDS of claim 23, wherein the AML or MDS are relapsed or refractory (R/R) AML or R/R higher-risk types of MDS.

25. The method of treating AML or MDS of claim 23, wherein the unit dose of the CD33×Vδ2 bispecific antibody is administered once every two weeks (Q2W), or once every week (Q1W).

26. The method of treating AML or MDS of claim 23, wherein the unit dose of the CD33×Vδ2 bispecific antibody is administered intravenously.

27. The method of treating AML or MDS of claim 23, wherein the unit dose of the CD33×Vδ2 bispecific antibody is administered by infusion during 0.5-4 hours, or about 1 hour.

28. The method of treating AML or MDS of claim 23, wherein the unit dose of the CD33×Vδ2 bispecific antibody is administered until progression, relapse, refractory response, intolerable toxicity, or continuous clinical benefit.

29.-34. (canceled)

35. A method of treating AML or MDS in a patient, said method comprising administering to the patient in need thereof, the pharmaceutical composition of claim 11.

36. The method of treating AML or MDS of claim 35, wherein the AML or MDS are relapsed or refractory (R/R) AML or R/R higher-risk types of MDS.

37. The method of treating AML or MDS of claim 35, wherein the pharmaceutical composition is administered once every two weeks (Q2W), or once every week (Q1W).

38. The method of treating AML or MDS of claim 35, wherein the pharmaceutical composition is administered intravenously.

39. The method of treating AML or MDS of claim 35, wherein the pharmaceutical composition is administered by infusion during 0.5-4 hours, or about 1 hour.

40. The method of treating AML or MDS of claim 35, wherein the pharmaceutical composition is administered until progression, relapse, refractory response, intolerable toxicity, or continuous clinical benefit.
